(19) United States
(12) Patent Application Publication
(10) Pub. No.: US 2008/0275139 A1
(43) Pub. Date: Nov. 6, 2008

(54) Cyclodextrin-Containing Polymers and Uses Thereof

(75) Inventor: Jallal M. Gnaim, Baka El Garbia (IL)

Correspondence Address: BROWDY AND NEIMARK, P.L.L.C.;624 NINTH STREET, NW, SUITE 300, WASHINGTON, DC, 20001-5303, US

(73) Assignee: Capsutech Ltd., Nazareth (IL)

(21) Appl. No.: 12/158,091

(22) PCT Filed: Dec. 19, 2006
(86) PCT No.: PCT/IL06/01459

§ 371 Date: Jun. 19, 2008

Related U.S. Application Data
(60) Provisional application No. 60/751,295, filed on Dec. 19, 2005.
(60) Provisional application No. 60/854,074, filed on Oct. 25, 2006.

(51) Int. Cl. A61K 47/26; C07K 4/00; C07K 14/00; C07H 21/00; A61K 47/42

(52) U.S. Cl. 514/773; 530/300; 530/350; 536/23.1; 514/777; 530/345; 530/402; 536/124

(57) ABSTRACT

The invention provides a cyclodextrin-containing polymer comprising one or more cyclodextrin residues. The polymer is selected from a peptide, a polypeptide, an oligonucleotide or a polynucleotide or a mixture thereof. The peptide or polypeptide has at least one amino acid residue containing a functional side group and at least one of the cyclodextrin residues is covalently linked to the functional side group of the amino acid residue of said peptide or polypeptide or to the sugar moiety of a nucleotide residue of the oligonucleotide or polynucleotide.

### FIELD OF THE INVENTION

The present invention relates to polymers containing cyclodextrins, more specifically to peptides and proteins containing cyclodextrin, to processes and intermediates for producing them and uses thereof.

### BACKGROUND OF THE INVENTION

There is a continuous need for an effective system that delivers bioactive materials at the site of action, while minimizing peak-trough fluctuations. Ideally such a system would eliminate undesirable side effects and reduce dosage and frequency of administration while improving visible effects.

Many technologies are already in place, including multiple emulsions, microemulsions, microspheres, nano-spheres, microsponges, liposomes, cyclodextrins, skin patches and unit dosages. Among all these technologies, some studies indicate that liposomes, microspheres and nano-spheres are most suitable for transferring cosmetic actives into the sub-epidermal level. Another convenient delivery method employs biodegradable polymeric matrices that deliver cosmetic macro or micromolecules onto or into the stratum corneum. Polymeric cosmetic conjugates can also be designed for specific target areas.

Effective delivery systems enable formulators to target specific skin maladies or conditions such as dryness or oiliness. The linkage between the actives may be loose or stable depending on chemical interaction (bonding) or loose affiliation such as surface adsorption or absorption on the polymer. A cosmetic active can be released onto the skin or within the stratum corneum by the cleavage of the cosmetic active and polymer chain link via hydrolysis or enzymatic degradation. This approach is especially suitable for delivering cosmetic actives such as vitamins, amino acids, peptides and lipids. Thanks to recent advancements in chemistry, the polymer cosmetic active can also be designed in such a way that only those enzymes present on the skin activate it.

There are manifold advantages of a biodegradable delivery system: maintenance of constant cosmetic active concentration for a desired time period at the target site, especially useful for providing moisturizers and other skin beneficial materials; improved product stability; reduced dosing time and an improved treatment effect; elimination or reduction of side effects that also includes irritation and precise local targeting to the site.

Microencapsulation is a growing field that is finding application in many technological disciplines, such as in the food, pharmaceutical, cosmetic, consumer and personal care products, agriculture, veterinary medicine, industrial chemicals, biotechnology, biomedical and sensor industries. A wide range of core materials has been encapsulated. These include adhesives, agrochemicals, catalysts, living cells, flavor oils, pharmaceuticals, vitamins, and water. There are many advantages to microencapsulation. Liquids can be handled as solids; odor or taste can be effectively masked in a food product; core substances can be protected from the deleterious effects of the surrounding environment; toxic materials can be safely handled; and drug delivery can be controlled and targeted. However, the microencapsulation technology has limited use for drug targeting, and poor water solubility (Orriols et al., 2005; Dai et al., 2005; International Food Ingredients, 2003).

Encapsulation also can occur on a molecular level. This can be accomplished, for example, by using a category of carbohydrates called cyclodextrins (CDs). Encapsulates made with these molecules may possibly hold the key for many future encapsulated formulation solutions. CDs are a general class of molecules composed of glucose units connected by α-1,4 glycosidic linkages to form a series of oligosaccharide rings. In nature, the enzymatic digestion of starch by CD glycosyltransferase (CGTase) produces a mixture of CDs comprised of 6, 7 and 8 glucose units, known as α-, β- and γ-CD, respectively, depicted below.

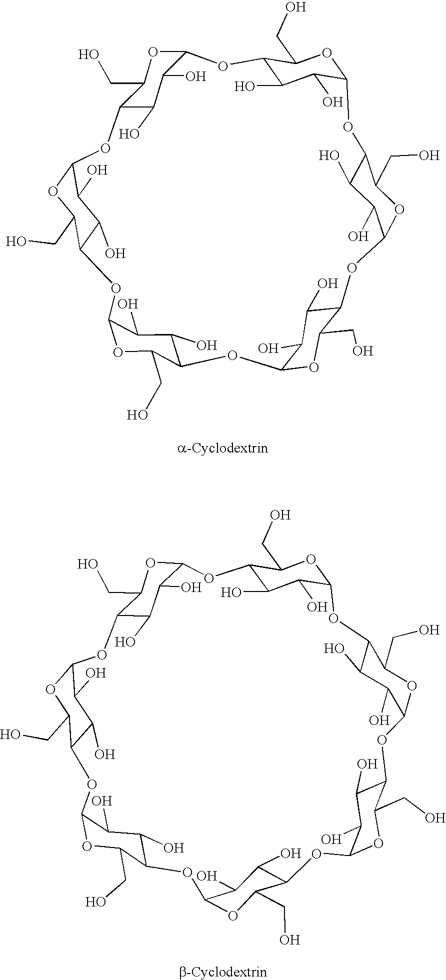

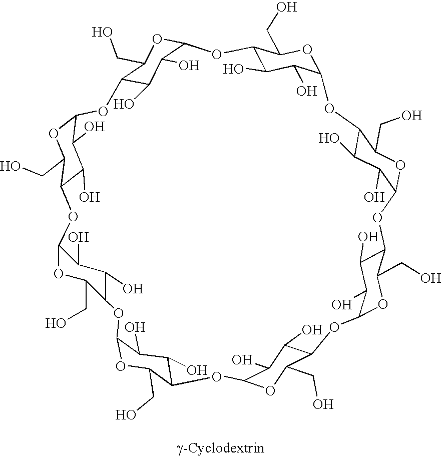

Commercially, cyclodextrins are still produced from starch, but more specific enzymes are used to selectively produce consistently pure α-, β- or γ-CD, as desired. All three cyclodextrins are thermally stable (<200° C.), biocompatible, exhibit good flow properties and handling characteristics and are very stable in alkaline (pH<14) and acidic solutions (pH>3).

As a result of their molecular structure and shape, the cyclodextrins possess a unique ability to act as molecular containers (molecular capsules) by entrapping guest molecules in their internal cavity. The ability of a cyclodextrin to form an inclusion complex with a guest molecule is a function of two key factors. The first is steric and depends on the relative size of the cyclodextrin to the size of the guest molecule. The second critical factor is the thermodynamic interactions between the different components of the system (cyclodextrin, guest, solvent). The resulting inclusion complexes offer a number of potential advantages in cosmetic and pharmaceutical formulations.

Molecular encapsulation is more comprehensive and much more controlled. For concentrated ingredients, this ability helps to assure an even dispersion in the final product. This control also helps saving on costly ingredients.

Shaped like a lampshade, the cyclodextrin molecule has a cavity in the middle that has a low polarity (hydrophobic cavity), while the outside has a high polarity (hydrophilic exterior). Since water is polar, cyclodextrin dissolves well in it. Forming a cyclodextrin complex can be as simple as mixing the cargo into a water solution of CD and then drawing off the water by evaporation or freeze-drying. The complex is so easily formed because the hydrophobic interior of the CD drives out the water through thermodynamic forces. The hydrophobic portions of the cargo molecule readily take the water's place.

As a result of their unique ability to form inclusion complexes, CDs provide a number of benefits in cosmetic and pharmaceutical formulations: bioavailability enhancement; active stabilization; odor or taste masking; compatibility improvement; material handling benefits; and irritation reduction. CDs have been used in Europe and Japan for many products (Duchene, 1987). Japanese manufacturers, in particular, have used them in hundreds of products during the past 15 years. In the United States, CD is used to remove the cholesterol from eggs (Li and Liu, 2003; Barse et al., 2003).

However, molecular encapsulation technology employing CDs suffers from several drawbacks such as limited capacity of the CD cavity, rapid release of the encapsulated active molecules under physiological conditions and low water solubility of the native β-CD. Therefore, there is still a strong need for a new class of materials which have combined advantages of both methods, namely, microencapsulation and molecular encapsulation.

U.S. Pat. No. 5,631,244 discloses a mono-6-amino-6-deoxy-β-CD derivative substituted in the 6-position by an α-amino acid residue and cosmetic or dermatological compositions comprising said CD derivative or an inclusion complex of said CD derivative and an active substance.

An object of the present invention is to provide a process for producing a highly water-soluble and biodegradable polymer containing cyclodextrins, which have improved stabilization, targeting and controlled release characteristics as well as a wider spectrum of applications with sizes and formulations.

### SUMMARY OF THE INVENTION

The present invention relates, in one aspect, to a CD-containing polymer comprising one or more CD residues, wherein said polymer is selected from a peptide, a polypeptide, an oligonucleotide or a polynucleotide. The peptide or polypeptide comprises at least one amino acid residue containing a functional side group. A CD residue is linked covalently to said functional side group or to the sugar moiety of a nucleotide residue of said oligonucleotide or polynucleotide.

In another aspect, the invention relates to compositions, including pharmaceutical and cosmetic compositions, comprising a CD-containing polymer of the invention and an active agent encapsulated within the cavities of the CD residues and/or embedded within the polymer matrix.

The active ingredient for encapsulation according to the invention may be organic or inorganic, natural or synthetic, substances such as, but not limited to, vitamins, natural extracts, individual compounds prepared synthetically or isolated from a natural source, pigments, fragrances, odor agents, color agents and volatile natural and synthetic compounds.

In a further aspect, the present invention relates to a method for combined microencapsulation and molecular encapsulation of an active agent in a sole carrier system which comprises reacting said active agent with a CD-containing polymer of the invention, whereby the active agent is microencapsulated and/or molecular encapsulated within said CD-containing polymer.

In still a further aspect, the invention provides cyclodextrin derivatives in which two cyclodextrin residues are covalently kinked to an amino acid selected from glutamic acid, aspartic and lysine, such compounds having an active agent encapsulated therein and compositions comprising them.

In yet a further aspect, the invention provides cyclodextrin derivatives linked at position 6 via a NH—CH₂—CH₂—NH— group to the functional side group of an α-amino acid selected from aspartic acid, glutamic acid, lysine, tyrosine, cysteine, serine, threonine and histidine.

### DETAILED DESCRIPTION OF THE INVENTION

The present invention provides a cyclodextrin (CD)-containing polymer comprising one or more CD residues, wherein said polymer is selected from a peptide, a polypeptide, an oligonucleotide or a polynucleotide. When the polymer is a peptide or polypeptide, it has at least one amino acid residue containing a functional side group and at least one of the CD residues is covalently linked to said functional side group. When the polymer is an oligonucleotide or a polynucleotide, at least one of the CD residue(s) is covalently linked to the sugar moiety of a nucleotide residue of said oligonucleotide or polynucleotide. The polymer may also be a polymer that is kind of a conjugate or a chimera of a peptide or a polypeptide with an oligonucleotide or a polynucleotide.

The present invention provides an innovative technology that will permit broader and more focused applications of the CD encapsulation technique. The technology provided by the present invention is based on coupling a natural or a chemically modified CD with a natural or synthetic polymer consisting of a peptide, a polypeptide, a protein, an oligonucleotide or a polynucleotide, or a combination thereof, which polymer serves to group together a predefined, controlled number of CD molecules in one capsulation.

The CD's unique shape and hydrophilic nature is beneficial for the inclusion and delivery of large, unstable molecules, and water-insoluble active ingredients. The formulation of the invention operates in the molecular- and/or nano-range, allowing for the inclusion of even one single molecule within the CD-containing polymer, and a predefined number of molecules in any capsulation.

The terms “active ingredient” or “active substance” or “active agent” are used herein interchangeably and refer to the material located within the cavity of the cyclodextrin moiety, and/or embedded within the CD-containing polymer matrix, which material may include one or more agents having biological activity, an odor agent or a color agent, and may include non-active ingredients such as a plasticizer, and the like.

In one preferred embodiment, the CD-containing polymer comprises a peptide or polypeptide as the backbone polymer and one or more CD residues, wherein at least one of the amino acid residues of said peptide or polypeptide has a functional side group and at least one of the CD residues is covalently linked to said functional side group. Other CD residues may be linked to different functional side groups of other amino acid residues in said peptide or polypeptide chain and one or two CD residues may be covalently linked to the α-amino- and/or α-carboxy-terminal groups of said peptide or polypeptide. It should be understood that if only one CD moiety is attached to a peptide or polypeptide polymer, it is not linked to a terminal amino or carboxy group of said peptide or polypeptide. In some embodiments, all the amino acids of the peptide have side-chain functional groups and are bound through their side-chain functional groups to CDs and, thus, said peptide has no free functional side groups.

The peptide or polypeptide may be an all-L or all-D or an L,D-peptide or polypeptide, in which the amino acids may be natural amino acids, non-natural amino acids and/or chemically modified amino acids. According to the present invention, at least one of the natural, non-natural or chemically modified amino acids of said peptide or polypeptide has a side-chain functional group.

In a more preferred embodiment, the peptide or polypeptide comprises only natural amino acids. The term “natural amino acid” refers to an amino acid selected from the 20 known natural amino acids. Natural amino acids having functional side group suitable for the purpose of the invention include lysine, aspartic acid, glutamic acid, cysteine, serine, threonine, tyrosine and histidine.

The peptide or polypeptide may, according to another preferred embodiment, comprise one or more non-natural amino acids such as, but not limited to, an N_{α}-methyl amino acid, a C_{α}-methyl amino acid, a β-methyl amino acid, β-alanine (β-Ala), norvaline (Nva), norleucine (Nle), 4-aminobutyric acid (γ-Abu), 2-aminoisobutyric acid (Aib), ornithine (Orn), 6-aminohexanoic acid (ε-Ahx), hydroxyproline (Hyp), sarcosine, citruline, cysteic acid, statine, aminoadipic acid, homoserine, homocysteine, 2-aminoadipic acid, diaminopropionic (Dap) acid, hydroxylysine, homovaline, homoleucine, 1,2,3,4-tetrahydroisoquinoline-3-carboxylic acid (TIC), naphthylalanine (NaI), and a ring-methylated or halogenated derivative of Phe.

The peptide or polypeptide may further comprise chemically modified amino acids. Examples of said chemical modifications include: (a) N-acyl derivatives of the amino terminal or of another free amino group, wherein the acyl group may be a C₂-C₂₀ alkanoyl group such as acetyl, propionyl, butyryl, hexanoyl, octanoyl, lauryl, stearyl, or an aroyl group, e.g., benzoyl; (b) esters of the carboxyl terminal or of other free carboxyl groups, for example, C₁-C₂₀ alkyl, phenyl or benzyl esters, or esters of hydroxy group(s), for example, with C₂-C₂₀ alkanoic acids or benzoic acid; and (c) amides of the carboxyl terminal or of another free carboxyl group(s) formed with ammonia or with amines.

In one embodiment of the invention, the peptide is an oligopeptide of 2-20, preferably, 2-10, 2-5, 2-3, more preferably, 2 amino acid residues. The oligopeptide may be a homooligopeptide that is composed of identical amino acid residues. In a more preferred embodiment, the oligopeptide is a homodipeptide, more preferably Glu-Glu, Asp-Asp, Lys-Lys or Cys-Cys. In more preferred embodiments, the CD-containing peptides are the polyglutamic acid peptides 24 and 26 and polyaspartic acid peptides 25 and 27 (Schemes 10 and 13) and the glutamic acid dipeptides 33 and 34 (Scheme 12).

In another embodiment, the polypeptide or protein has 21 to 10,000, preferably, 100-1,000 or 100-500 amino acid residues. In a more preferred embodiment, the polypeptide is a homopolypeptide of an amino acid having a functional side group such as α- or ε-polylysine, α- or γ-polyglutamic acid, α- or β-polyaspartic acid, polycysteine, polyserine, polythreonine or polytyrosine. These polypeptides are commercially available.

According to other embodiments, the polypeptide is a synthetic random copolymer of different amino acids, wherein at least one of the amino acids has a functional side group, or it is a native, preferably inert, protein such as albumin, collagen, an enzyme such as a collagenase, a matrix metalloproteinase (MMPs) or a protein kinase such as Src, v-Src, a growth factor, or a protein fragment such as epidermal growth factor (EGF) fragment.

As used herein, the term “protein” refers to the complete biological molecule having a three-dimensional structure and biological activity, while the term “polypeptide” refers to any single linear chain of amino acids, usually regardless of length, and having no defined tertiary structure.

The peptide or polypeptide used in the invention to form the CD-containing polymer may also be covalently linked to a carbohydrate residue to form a glycopeptide, a glycopolypeptide or a glycoprotein. The carbohydrate residue may be derived from a monosaccharide such as D-glucose, D-fructose, D-galactose, D-mannose, D-xylose, D-ribose, and the like; a disaccharide such as sucrose and lactose; an oligo- or polysaccharide; or carbohydrate derivatives such as esters, ethers, aminated, amidated, sulfated or phospho-substituted carbohydrates. The glycopolypeptide may contain one or more carbohydrate residues. Some glycoproteins contain oligosaccharide residues comprising 2-10 monosaccharide units. The carbohydrate may be linked via a free amino group or carboxy group in the side chain of an amino acid residue, e.g., lysine, glutamic acid or aspartic acid, forming an N-glycosyl linkage with the carbohydrate, or via a free hydroxyl group of an amino acid residue, e.g., serine, threonine, hydroxylysine or hydroxyproline, forming a O-glycosyl linkage with the carbohydrate. The glycopeptides and glycopolypeptides can be obtained by enzymatic or chemical cleavage of glycoproteins, or by chemical or enzymatic synthesis as well known in the art.

Examples of glycoproteins useful according to the invention as components of the CD-containing polymer include collagens, fish antifreeze glycoproteins, lectins, hormones such as follicle stimulating hormone, luteinizing hormone, thyroid stimulating hormone, human chorionic gonadotropin, alpha-fetoprotein and erythropoietin (EPO), and proteoglycans (known also as glycosaminoglycans).

In other embodiments, the polymer consists of an oligonucleotide that may be a ribonucleotide or a deoxyribonucleotide oligonucleotide containing from 2 to 25 bases or the polymer is a ribonucleotide or a deoxyribonucleotide polynucleotide containing 26-1000 bases or more.

The CD in the CD-containing polymer of the invention may be a natural CD selected from α-, β- and/or γ-CD or a CD derivative. When two or more CD residues are linked to the polymer, they can be identical or different. For example, the CD-containing polymer may comprise both α- and β-CD residues or any other combination of α-, β- and/or γ-CD residues. In preferred embodiments, the CD-containing polymer comprises only β-CD residues.

As used herein the terms “modified cyclodextrin” or “modified CD” are used interchangeably and refer to a cyclodextrin molecule which was chemically modified in order to facilitate its bonding to a side chain of an amino acid prior to polymerization, or to an amino acid of the polymer backbone. As described herein, this modification is carried out by replacing one hydroxyl group, preferably at position 6, of the CD molecule with a group —NH₂, —NH(CH₂)ₘNH₂, —SH, —O(CH₂)ₘCOOH, —OC(O)(CH₂)ₘCOOH, —NH(CH₂)ₘCOOH, —OC(O)(CH₂)ₘNH₂, —Br, —Cl, —I, or —OSO₂Ar, wherein Ar is a (C₆-C₁₄) aryl, preferably phenyl, and m is 1, 2, 3, 4 or 5.

Any cyclodextrin derivative which has at least one free hydroxyl group at position 6 or 2 or 3, preferably position 6 (and thus can be modified as described above), can be used in the invention. These derivatives include, but are not limited to, acetyl-β-CD, diacetyl-β-CD, carboxymethyl-β-CD, methyl-β-CD, dimethyl-β-CD, partially methylated-β-CD (i.e., cyclodextrins wherein the hydroxyl groups in position 2 or 6 are not fully replaced by methyl groups), 2-hydroxyethyl-β-CD, 2-hydroxypropyl-β-CD, 2-hydroxyisobutyl-β-CD, 2-hydroxypropyl-γ-CD and β-CD sulfobutyl ether sodium salt. These CD derivatives are usually much more soluble than the native β-CD. In addition, the derivatives formed by substitution with hydroxyalkyl groups have reduced toxicity and optimized solvent action.

When the CD-containing polymer of the invention contains a CD derivative, it can be prepared starting with a modified CD derivative that is grafted onto the polymer or, alternatively, the chemical modification of CD is carried out after grafting same onto a polymer.

Cyclodextrin hosts are capable of forming inclusion complexes by encapsulating guest molecules within their cavity, thus greatly modifying the physical and chemical properties of the guest molecule, mostly in terms of water solubility and chemical stability. Since the CDs are cyclic oligosaccharides containing 6-8 glucopyranoside units, they can be topologically represented as toroids (or doughnuts) wherein the larger and the smaller openings of the toroid (the secondary and primary hydroxyl groups, respectively) are exposed to the solvent. Because of this arrangement, the interior of the toroids is not hydrophobic, but considerably less hydrophilic than the aqueous environment and thus is able to host other hydrophobic molecules. On the other hand, the exterior is sufficiently hydrophilic to impart cyclodextrins (or their complexes) water solubility.

The CD-containing polymer of the invention is a system useful for the delivery of one or more kinds of active agents, for increasing the water solubility and improving the stability of water-insoluble active agents and/or as a mean for controlled release of the active agents. This system combines two categories of encapsulation: molecular encapsulation and microencapsulation. The CD residues attached to the polymer backbone serve as molecular encapsulators such that each CD residue (the host) forms an inclusion complex with a part of one molecule or with a whole molecule or with more than one molecule of the active agent (the guest). In addition, the polymer matrix as a whole can microencapsulate the active agent by embedding or entrapping molecules of the active agent within the matrix.

Thus, in one preferred embodiment, the CD-containing polymer of the invention contains an active ingredient encapsulated within the cavity of the cyclodextrin residues. In another preferred embodiment, the active ingredient is further entrapped and/or embedded, i.e., microencapsulated, within the CD-containing polymer matrix.

As used herein, the term “an active ingredient” or “active agent” are used interchangeably and refer to a sole active ingredient or agent or to more than one active ingredient or agent.

The present invention, thus, further provides a method for combined micro- and molecular-encapsulation of an active agent in a sole carrier, said method comprises contacting (i.e., mixing, blending) said active agent with a CD-containing polymer, whereby the active agent is both encapsulated and entrapped within said cyclodextrin-containing polymer.

When the polymer is a peptide or polypeptide, controlled release of an active ingredient is triggered by the enzymatic degradation (enzymatic hydrolysis or dissociation) of the peptide or polypeptide, as they encounter specific enzymes at the target site. The hydrolyzing/digesting enzymes include all the proteases (proteinases, peptidases or proteolytic enzymes) that break peptide bonds between amino acids of proteins by proteolytic cleavage, a common mechanism of activation or inactivation of enzymes especially involved in blood coagulation or digestion. There are currently six classes of proteases: serine proteases, threonine proteases, cysteine proteases, aspartic acid proteases (e.g. plasmepsin), metalloproteases and glutamic acid proteases. The different proteases depend on the peptide or polypeptide sequence. Thus, chymotrypsin is responsible for cleaving peptide bonds following a bulky hydrophobic amino acid residue, preferably phenylalanine, tryptophan and tyrosine, which fit into a snug hydrophobic pocket. Trypsin is responsible for cleaving peptide bonds following a positively-charged amino acid residue. Instead of having the hydrophobic pocket of the chymotrypsin, there exists an aspartic acid residue at the base of the pocket. This can then interact with positively-charged residues such as arginine and lysine on the substrate peptide to be cleaved. Elastase is responsible for cleaving peptide bonds following a small neutral amino acid residue, such as alanine, glycine and valine.

The dissociation of the peptide by the protease leads primarily to release of microencapsulated molecules, i.e. molecules embedded within the polymer matrix, and thus activates a first pulse of active ingredient release. This is followed by slow release, mainly of molecules encapsulated within the CDs. These advantages may be utilized to achieve unique effects in the design of a wide variety of pharmaceutical and cosmetic applications. They include controlled release of active ingredients performed in two stages: (i) an initial pulse, releasing a substantial dose of the active ingredient, thus achieving an immediate effect; and (ii) continuous, controlled release, providing a prolonged effect of the active ingredient, over a, preferably predefined, number of hours.

The CD-containing polymer of the invention also allows for a masking effect of undesired odor, color or taste or, alternatively, controlled release of desirable scent or color.

The technology of the present invention is beneficial also in targeted drug delivery of multiple drug molecules, to treat a variety of medical conditions. The unique structure and qualities of the capsulation according to the invention offers the following unique benefits: (i) increased stability for large, unstable molecules such as insulin, allowing for a wider range of drug administration methods such as oral; (ii) delivery of water-insoluble active ingredients such as steroids; (iii) prevention of adverse effects by capsulated delivery to the target site, for example, with anti-cancer chemotherapy drugs or antibiotics; (iv) highly specific targeting enabled by complexing the CD-containing polymers with additional ingredients, known to improve specificity and cell permeability such as hormones, antibodies or sugars; and (v) prevention of a contrast effect between drugs or other biologically active substances.

According to the present invention, one or more kinds of active ingredients can be encapsulated and delivered simultaneously. Thus, for example, when the CD-containing polymer comprises two types of CD residues e.g., α- and β-CD, two kinds of active ingredients, which differ in molecular size, can be encapsulated within the same polymer. First, the larger molecules are contacted with the CD-containing polymer, resulting in occupation of the larger cavities of β-CD. Then, this CD-containing polymer is contacted with the smaller molecules, which are encapsulated by the smaller α-CD residues.

In one aspect of the present invention, the CD-containing polymers are used as such, namely without encapsulated active agent, especially as moisturizing agents in cosmetic or dermatological composition, for example, for treatment of skin or hair. According to this embodiment, the CD-containing polymers are generally presented at a concentration of between 0.1%-20% by weight with respect to the total weight of the composition.

In another aspect of the present invention, the CD-containing polymers are used for the encapsulation and delivery of active ingredients. These active ingredients are preferably substances of lipophilic, hydrophobic or amphiphilic nature, or are substances insoluble or unstable in aqueous medium. In a more preferred embodiment, said active ingredients comprise at least one agent having biological activity, an odor agent or a color agent, and are more particularly chosen from the active ingredients, e.g., drugs, cosmetic and cosmeticeutical agents used in the cosmetic, dermatological, pharmaceutical and food fields. Active ingredients applicable according to the invention include, for example but are not limited to, the compounds described in U.S. Pat. No. 5,631,244, incorporated herein by reference in its entirety as if fully described herein, and are selected from: anti-oxidizing agents and compounds which act against free radicals such as vitamins; anti-acne, anti-aging or anti-photoaging agents such as retinoic acid and its isomers, retinol and its esters; agents for controlling psoriasis such as anthralin, psoralens or aromatic retinoids; agents promoting hair growth or preventing hair loss; hair dyes which are difficult to dissolve in aqueous media or unstable dyes; agents for hydrating and/or plasticizing the stratum corneum such as α-hydroxyacids, thiamorpholine derivatives; agents for reconstituting the lipid barrier such as ceramides and their derivatives; depigmenting agents such as hydroquinone; sunscreening agents; anti-inflammatory agents; preserving and bactericidal agents, such as substituted isothiazolones; steroids; anti-viral and anti-cancer agents.

Metal salts can also be encapsulated according to the invention, especially metal salts active in the oxidation-reduction process during hair dyeing or bleaching or during dyeing or controlling aging of the skin.

Vitamins that can be encapsulated according to the invention include the vitamins A, B, C, D, E, F, K, P, or mixtures thereof.

In one embodiment, the vitamin is vitamin A, either in its free form as retinol or in its ester form as retinol palmitate. The most useable form of the vitamin is retinol, the active form in the body. Retinol is an anti-oxidant vitamin used as nutritional factor and also as an active ingredient of topical/dental products. Retinol can be used for topical treatment of Ichthyosis vulgaris (an inherited skin disorder characterized by cornification of the skin) and common acne, and in anti-aging and rejuvenation formulations. However, retinol (an unsaturated alcohol) is a small and unstable molecule and undergoes chemical degradation/oxidation due to its high potential for chemical reactions with other molecules and should be stabilized before using it as an active ingredient in compositions. In order to enjoy the beneficial effects of retinol and meet the shelf-life requirements needed for topical/dental compositions, this active principle should be protected from oxidation. Encapsulation of retinol by the CD-containing polymer of the invention provides an effective solution for its stabilization and protection. The encapsulated retinol according to the invention is highly compatible with all types of topical/dental formulations and can be used in various applications including, without limiting, dental products, anti-aging products (creams, lotions, serums and masks), skin regeneration formulations, nourishing and moisturizing creams and anti-acne products.

In another embodiment, the vitamin is vitamin C (ascorbic acid), used in recent years as an active ingredient of cosmetics. Due to its antioxidant properties, it is considered to confer both antioxidant and photoprotection to skin against free radical attack and UV ray damage. However, vitamin C is easily oxidized and, upon storage, exposure to light, oxygen, moisture and/or high temperature, undergoes rapid degradation. It is unstable in aqueous solution, even under neutral pH and at room temperature. The molecular encapsulation of vitamin C by the CD-containing polymer of the present invention permits its use as active ingredient in cosmetic composition for use as moisturizing cream, anti-aging cream, anti-wrinkle cream, sunscreen cream, and for stimulating collagen production.

In a further preferred embodiment, the vitamin is vitamin E, preferably as α-tocopherol. Tocopherols are well-known for their antioxidant properties making vitamin E one of the most widely consumed vitamins. However, vitamin E in its ester form (e.g., tocopherol acetate) is only effective as antioxidant to the formulation, but not to the skin because it is inherently unstable. The CD-containing polymer of the invention encapsulates stable α-tocopherol, and can be used in various types of cosmetic formulations such as sunscreen products, shampoos, conditioners, hair gels, liquid make-up and make-up tissue remover, and release about 95-97% of vitamin E directly onto the skin/scalp upon application.

In a further embodiment, the vitamin is vitamin F, a mixture of unsaturated fatty acids essential for skin health and functionality, also known as Essential Fatty Acids (EFA; linoleic acid and alpha-linolenic acid.). Vitamin F undergoes rapid oxidation when incorporated in cosmetic formulation. The microencapsulation/molecular encapsulation with the CD-containing polymers of the invention offers a stable, active and odorless system of vitamin F suitable for incorporation into moisturizing creams, anti-aging agents and anti-dryness serums.

In another embodiment, the vitamin is rutin (quercetin-3-rutinoside or vitamin P1), one of the most active natural flavanoids, highly effective as an antioxidant and free radical scavenger and in the treatment of cellulite due to its ability to control cross-linking of collagen synthesis. Rutin is widely applied in dermatological and cosmetic products due to its beneficial effects on the appearance of healthy skin and is well known for its potent antioxidant and anti-inflammatory properties and ability to strengthen and modulate the permeability of the walls of the blood vessels including capillaries. However, when incorporated into cosmetic formulations in its non-encapsulated form, rutin tends to react with other ingredients and oxidizes quickly, resulting in change of the original color of the formulation and loss of its original biological activity. In order to maintain its potent biological activity and prevent its oxidation in cosmetic formulations, rutin should be stabilized. CD-containing polymers encapsulating rutin are developed according to the present invention, specifically for topical application in order to stabilize the rutin, preferably containing a high concentration (about 7%) of pure rutin hydrate from plant source.

In another embodiment of the present invention, the active ingredient having biological activity is a compound present in a natural extract. In cosmetics, a natural extract is assumed to mean ingredients of botanical origin. To be truly natural it must be extracted from the relevant part of the plant without undergoing any significant chemical change. Any compound isolated from herbal extract used for topical application, for example in the cosmetic industry, can be used according to the invention, but preferred herbal extracts for encapsulation according to the invention include Licorice root extract and grape juice extract.

Thus, in one preferred embodiment of the invention, the natural extract is extracted from grape juice, which contains a high content of proanthocyanidins (also known as Oligomeric Proanthocyanidin Complexes or OPCs), a class of nutrients that belong to the flavonoid family and are potent antioxidants and free radical scavengers, reducing the harmful effects of UV radiation. In topical use, a great advantage of OPCs is a substantial increase in blood circulation at the sub-epitopical level and an improvement of intracellular membrane exchange of micronutrients. The OPCs, however, are not stable and oxidize rapidly due to temperature and light influence or cross-reactions with other ingredients of topical formulation. The brown color developed in the final product is a result of OPCs oxidation. Encapsulation of OPCs according to the present invention prevents oxidative degradation and brown color development, since the polymeric microcapsulation and/or the CD molecular encapsulation prevent interaction of OPCs with other ingredients of the formulation, as well as guarantees the maximum release of OPCs on the skin upon application with maximum biological affect. OPCs are thus indicated as an active ingredient for incorporation in anti-aging creams, in after-sun creams for reduction of skin erythema, in moisturizing and revitalizing products, and in facial sunscreens for prevention of UV-induced lipid oxidation in skin.

In another preferred embodiment, the biologically active ingredient is glabridin, present in natural extract of Licorice root. Glabridin is a flavanoid known for its beneficial effects on the skin due to its anti-inflammatory and antioxidant properties. In addition, glabridin has whitening/lightening and anti-spot properties, probably due to inhibition of tyrosinase and melanin synthesis. However, this compound tends to oxidize easily, resulting in a loss of glabridin's original whitening activity. Moreover, glabridin, as a flavanoid, is sensitive to pH changes and this factor is the reason for extreme instability of glabridin in topical formulations, resulting in loss of its original activity and in the development of a dark brown color in formulations. The CD-containing polymers of the present invention encapsulating glabridin, provide stable lightening/whitening agent, prevent oxidation of the glabridin, thereby guaranteeing original activity of glabridin and providing a longer shelf life of the end product; prevent development of brown color in formulations; are highly stable in a wide pH range; are freely dispersible in all types of cosmetic formulations; and provide a unique control release of the biologically active ingredient only upon application onto the skin. These glabridin-encapsulating polymers of the invention are, therefore, indicated as an active ingredient in whitening creams and lotions, age-defying creams and serums, anti-spots treatment formulations and lightening hand creams.

In a further embodiment of the invention, the active substance to be encapsulated is an individual compound isolated from a natural source such as, but not limited to, a coumarin, a chalcone or a flavonoid selected from the group consisting of flavans, flavanols, flavonols, flavones, flavanones, isoflavones, anthocyanidins, and proanthocyanidins.

It should be understood that an active ingredient used in the present invention may belong to more than one category as defined herein. Thus, rutin, defined above as vitamin P, is a flavonoid, as well as glabridin of the Licorice root extract and the proanthocyanidins of the grape juice extract.

In an additional embodiment of the invention, the active ingredient to be encapsulated is a pharmaceutical agent for topical applications, e.g. an antibiotic such as, but not limited to, a macrolide antibiotic selected from Erythromycin, Azithromycin or Clarithromycin. Clarithromycin is a semi-synthetic macrolide antibiotic used to treat certain infections caused by bacteria, such as pneumonia, bronchitis, and infections of the ears, lungs, sinuses, skin, and throat. It also is used to prevent disseminated Mycobacterium avium complex (MAC) infection in patients with human immunodeficiency virus (HIV). Clarithromycin is used orally, but expanding its use for topical application opens new possibilities for administration of this highly potent antibacterial agent with less tolerated drugs such as the tretinoins. Clarithromycin, as many other antibiotics, is very sensitive to degradation due to hydrolysis in water-containing formulations. The CD-containing polymers encapsulating Clarithromycin are specifically developed according to the invention for topical use and provide protection to the antibiotic from degradation when used in water-containing formulations.

In another embodiment of the invention, the active ingredient to be encapsulated is an odor (usually a pleasant odor) agent selected from the group consisting of fragrances, perfumes, essential oils and compounds extracted therefrom, and volatile natural and synthetic compounds. These agents can be used to impart a pleasant odor to the cosmetic formulation and/or to mask an undesired odor of other components of the formulation. In a preferred embodiment, the active substance is an essential oil selected from thymol, carvacrol, eucaliptol, cinnamaldehyde, eugenol, menthol, cuminal, anethole, estragole, citronnellal, carvone, menthone, limonene, isoeugenol, bisabolol, camphor, geraniol, citral, and/or mixtures thereof.

Agents with odor properties are widely used in topical products. Typically, these agents such as fragrances, perfumes and other volatile materials suffer from instability under specific conditions such as pH of the formulation or they cross-react with other ingredients of the formulation. For these reasons, it is necessary to encapsulate this type of ingredients. The microcapsulating/molecular encapsulating CD-containing polymers of the invention containing a fragrance are developed specifically in order to solve the above-mentioned problems.

In one preferred embodiment, the volatile compound is menthol, a monocyclic terpene alcohol obtained from peppermint oil or other mint oils, or prepared synthetically by hydrogenation of thymol. Menthol is a white crystal with a characteristic refreshing mint odor, which provides cosmetic formulations with a fresh sensation, cooling effect, calming qualities and short-term relief. However, menthol, as a volatile ingredient, has a tendency to evaporate and to change the original content/odor of the formulation. In addition, it is difficult to disperse menthol homogeneously in cosmetic formulations and usually requires predispersion with ethanol. The precipitation of menthol from the formulations, its original strong characteristic odor and its potential cross-linking with other ingredients, are reasons that makes it difficult to use it in topical/dental products. The CD-containing polymers encapsulating menthol of the present invention are odorless, protect the menthol from oxidation and maintain its original activity after incorporation into cosmetic formulations. They mask menthol's characteristic odor while maintaining the original smell, preventing it from reacting with other ingredients in the formulation and providing a long lasting sensation/cooling effect upon application on skin. The CD-containing polymers are homogeneously dispersed in cosmetic formulations without requiring the use of alcohol and are, therefore, indicated as an ingredient for oral hygiene care, e.g. toothpastes, mouth rinses, sun-screen products, cooling after-sun lotions, calming creams and refreshing pre- and after-shave products.

In an additional embodiment of the invention, the active ingredient to be encapsulated is a color agent selected from the group consisting of organic and inorganic pigments, colorants and color agents from natural source.

Color agents that can be used according to the invention include the pigments carmine, iron oxides, titanium dioxide, and chrome oxide/hydroxide, the colorants D&C Red 21 Aluminum Lake, D&C Red 7 Calcium Lake, D&C Green 6 Liposoluble, and Aluminium Blue #1 (Indigo Carmine Lake). In one preferred embodiment, the pigment is titanium dioxide (used to lighten other pigments and to lend opacity to formulations) in any one of its mineral forms anatase, brookite or rutile, or mixtures thereof. In another preferred embodiment, the color agent is iron oxides, the most widely used of the inorganic pigments, in any of the 3 basic colors—red, black and yellow iron oxides, or mixtures thereof. From these 3 oxides and the addition of titanium dioxide, any shade of brown (skin tones) can be achieved.

Thus, in another aspect, the present invention provides compositions comprising an active agent encapsulated within the CD-containing polymer of the invention.

In one preferred embodiment, a pharmaceutical composition is provided, comprising a biologically active agent encapsulated within the CD cavity and/or entrapped within the CD-containing polymer.

In another preferred embodiment, the present invention provides a cosmetic or dermatological composition comprising an active agent encapsulated within the CD cavity and/or entrapped within the CD-containing polymer.

According to a further aspect of the present invention, a carrier is provided consisting of a CD-containing polymer as described above for controlled release of water-insoluble or unstable active agents.

The carrier of the invention is a platform that operates on nano-scale standards thus enabling the transfer and delivery of nanoparticles of active ingredients. This carrier, encapsulating nanoparticles of one or more active ingredients, can be formulated into various formulations or compositions along with suitable excipients.

Thus, according to a further aspect of the invention, a composition for controlled release of water-insoluble or unstable active ingredient(s) is provided, comprising nanoparticles of said active ingredient(s) encapsulated and/or entrapped within a CD-containing polymer.

In another further aspect, the present invention relates to cosmetic, pharmaceutical and dermatological compositions comprising the CD-containing polymers of the invention. These compositions can be provided in various forms, especially in the form of aqueous or aqueous/alcoholic lotions, gels or dispersions. The active ingredient-containing polymer is present in a proportion of 0.1% to 50% by weight.

The CD-containing polymers of the invention may be useful in a number of applications in a wide range of fields, other than the above mentioned cosmetic and pharmaceutical applications for drug release. For example, these polymers can be employed in environmental protection: they can effectively immobilize inside the CD cavities toxic compounds like trichloroethane or heavy metals, or can form complexes with stable toxic substances, like trichlorfon (an organophosphorus insecticide) or with sewage sludge, enhancing their decomposition.

In the food industry, the CD-containing polymers of the invention may be employed in the preparation of cholesterol-free products: the bulky and hydrophobic cholesterol molecule is easily lodged inside the cavities of the CD residues, then the polymers are removed, leaving behind a “low fat” food. Other food applications include the ability to stabilize volatile or unstable compounds and to reduce unwanted tastes and odour. The strong ability of complexing fragrances can also be used for other purposes. For example, first the CD-containing polymers can be exposed to a controlled contact with fumes of active compounds, whereby the active compounds are entrapped or encapsulated within the CD cavities. Then, the resulting encapsulated product can be added to fabrics or paper products. Such products are capable of releasing the fragrances during ironing or when heated, for example in a typical clothes dryer, thus releasing the fragrance into the clothes.

The present invention provides, in another aspect, processes for producing the CD-containing polymers of the invention.

In one preferred embodiment, the process comprises a first step of modification of the CD prior to its bonding to a functional side group of an amino acid, as depicted schematically in Schemes 1-3 herein. Thus, according to this preferred embodiment, the preparation of a modified CD may be carried out by replacement of one or more hydroxyl groups (—OH) at position 2, 3 and/or 6 with one or more functional groups Z selected from —NH₂, —NH(CH₂)ₘNH₂, —SH, —O(CH₂)ₘCOOH, —OC(O)(CH₂)ₘCOOH, —NH(CH₂)ₘCOOH, —OC(O)(CH₂)ₘNH₂, halogen such as Cl, Br or I, or —OSO₂Ar, wherein Ar is a (C6-C10) aryl, preferably phenyl, and m is 1, 2, 3, 4 or 5, as depicted in Scheme 1. The procedures below are suitable for all the three CDs but are exemplified for β-CD.

In a more preferred embodiment, the 6-hydroxyl group is replaced with an amino group to obtain the compound mono-6-deoxy-6-amino-β-CD, herein designated compound 4, as depicted in Scheme 2.

The mono-amino-β-CD derivative 4 is a known compound and can be prepared according to methods known in the art, for example as described in U.S. Pat. No. 5,068,227 or by other synthesis protocols, e.g. as described by Parrot-Lopez et al., 1990a, 1990b, 1990c, Takahashi et al., 1991. According to a more preferred embodiment, the derivative 4 can be prepared by reacting β-CD with p-toluenesulfonyl chloride (tosyl chloride, TsCl) to obtain the mono-tosyl-β-CD, herein designated compound 2, followed by reaction with NaN₃ to give the mono-azido-β-CD derivative (herein compound 3), and reduction of the azido derivative, for example with triphenyl phosphine/NH₃, thus, yielding the mono-amino-β-CD 4 (Scheme 2). According to an alternative embodiment, 4 may be obtained directly from the mono-tosyl-β-CD 2 by reaction with concentrated NH₄OH solution.

In another preferred embodiment, the hydroxyl of β-CD is replaced with ethylenediamino group to obtain the compound mono-6-deoxy-6-(2-aminoethyl)amino-β-CD, herein designated compound 5. As depicted in Scheme 3, β-CD is reacted with tosyl chloride and the obtained mono-tosyl-β-CD is then reacted with 1,2-diaminoethane/triethylamine reagent or with neat 1,2-diaminoethane, yielding the CD derivative 5.

In another preferred embodiment, an unmodified CD, herein termed “native CD”, is directly bonded to a free carboxy group of a functional side group of an amino acid through its OH group at position 6 or 3 or 2.

When the backbone polymer is a peptide or a polypeptide, the CD-containing polymer can be prepared using one of three alternative approaches or methods:

(i) covalently linking a native CD or modified CD to the free functional side group of a diprotected amino acid residue X—CH—(COOR₁)(NHR₂), wherein the amino acid may be aspartic acid, glutamic acid, serine, tyrosine, lysine, cysteine, and the like, to give the CD-amino acid derivative, as depicted in Scheme 4, followed by deprotecting the obtained derivative and polymerizing same to give the corresponding CD-containing peptide or polypeptide, as shown in Scheme 5;

(ii) covalently grafting a native CD or modified CD directly to one or more functional side groups of amino acids of a desired peptide, polypeptide or protein chain, as shown in Scheme 6. For a polypeptide of 300-400 amino acids, this process may result in 30-40% of random CD binding to the peptide backbone; or

(iii) coupling a free α-amino group of a CD-amino acid derivative with a free α-carboxy group of a second CD-amino acid derivative to give the corresponding CD-containing dipeptide as shown in Scheme 7. This method is suitable for the preparation of CD-containing oligopeptides of up to 10 amino acid residues, preferably 4, more preferably 2 amino acid residues, wherein each of the amino acids in the oligopeptide is covalently bound to a CD residue through its functional side group.

Diprotection of amino acids can be effected by blocking the α-amino and α-carboxy groups using approaches known in the art. Thus, the amino group may be blocked by tert-butyloxycarbonyl (t-Boc) or benzyloxycarbonyl protecting group, and the free carboxy group may be converted to an ester group e.g. methyl, ethyl, tert-butyl or benzyl ester.

Deprotection of the α-amino and α-carboxy groups is usually carried out under conditions that depend on the nature of the protecting groups used. Thus, benzyloxycarbonyl and benzyl groups are displaced by hydrogenation in the presence of Pd/C, and t-Boc groups are cleaved in the presence of trifluoroacetic acid at room temperature. The methyl, ethyl, tert-butyl or benzyl ester groups may be removed by saponification in the presence of sodium hydroxide (NaOH) solution or concentrated ammonium hydroxide (NH₄OH) solution.

Polymerization can be performed according to any suitable process known in the art for peptide polymerization. Prior to polymerization, either the α-amino or the α-carboxy group is protected, thus controlling the direction of peptide bond formation and the nature of the polymer synthesized. Homo- and hetero-polymers can be obtained using the same polymerization process. The resulting polymer's identity and length are determined by the kind and amount of amino acids introduced into the reaction batch and depend on the polymerization reaction conditions such as concentration of the reactants, reaction temperature, and stirring rate.

When different amino acids are employed in the polymerization process, a mixture of different peptides is obtained. These peptides differ in constitution and size. In the polymerization of homopeptides, peptides of different sizes are obtained. The peptides—homo- or heteropeptides, are separated based on their molecular size or weight using filtration means well known in industrial polymerization processes. For example, fractional isolation and purification of the peptides mixture may be carried out using a suitable membrane such that peptides having a given range of molecular weights are isolated depending on the pore size of the membrane.

In one preferred embodiment of the present invention, a CD-containing peptide or polypeptide is prepared according to method (i) above by a process comprising the steps of:

(i) preparing a modified CD by replacing at least one of the hydroxyl groups at position 6 or 3 or 2 with a functional group selected from —NH₂, —NH(CH₂)ₘNH₂, —SH, —O(CH₂)ₘCOOH, —OC(O)(CH₂)ₘCOOH, —NH(CH₂)ₘCOOH, —OC(O)(CH₂)ₘNH₂, —Br, —Cl, —I, or —OSO₂(C6-C10) aryl, preferably phenyl, wherein m is 1, 2, 3, 4, or 5;

(ii) covalently linking the modified CD to a side functional group of a diprotected amino acid residue to form a CD-amino acid derivative;

(iii) deprotecting the α-amino and/or α-carboxy of said CD-amino acid derivative; and

(iv) polymerizing said CD-amino acid derivative to obtain the desired CD-containing peptide or polypeptide.

In another preferred embodiment of the invention, the method (i) above is carried out with a native CD.

In a more preferred embodiment, the method (i) of the present invention is used for the production of CD-containing homopeptides. More preferably, the peptide is an oligopeptide comprised of glutamic-acid-CD or aspartic acid-CD monomers such as the herein designated homo-oligopeptides 24-27.

In another preferred embodiment, a CD-containing peptide, polypeptide or protein is produced according to method (ii) above by covalently grafting a native CD or modified CD directly to one or more functional side groups of amino acids of a desired peptide, polypeptide or protein chain. In a most preferred embodiment, the method (ii) is used for alografting mono-amino- and ethylenediamino-CD derivatives to polyglutamic acid or polyaspartic acid to obtain CD-containing polypeptides.

In yet another preferred embodiment, the present invention provides a process for producing a dipeptide according to method (iii) above, comprising the steps of:

(i) preparing a modified CD as described above;

(ii) covalently linking the modified CD to a side functional group of a first diprotected amino acid residue to form a CD-amino acid derivative (a); and

(iii) covalently linking a modified CD to a side functional group of a second diprotected amino acid residue to form a second CD-amino acid derivative (b); and

(iv) coupling the free α-amino of the CD-amino acid compound (a) with the free α-carboxy of the CD-amino acid compound (b) to obtain the desired CD-containing dipeptide.

In another preferred embodiment, the di-coupling method (iii) above is carried out with native CDs.

The di-coupling method (iii) of the present invention is preferably used for the production of CD-dipeptides, more preferably CD-homo-dipeptides, most preferably the β-CD-Glu-Glu derivatives, herein identified dipeptides 33 and 34.

In more preferred embodiments of methods (i) and (iii), in step (ii), the amino acid-CD derivative is obtained by reacting an α-amino acid selected from glutamic acid, aspartic acid, lysine or arginine, most preferably glutamic or aspartic acid, in the L, D or racemic form with a native or modified CD in an organic solvent such as dimethylformamide (DMF) or dimethylsulfoxide (DMSO) or a mixture of DMF and DMSO in the presence of an excess of a dehydrating agent such as dicyclohexylcarbodiimide (DCC) and a catalyst such as 1-hydroxybenzotriazole (HOBT), pyridine, 4-dimethylaminopyridine (DMAP), triethylamine or zeolite. The reaction is generally carried out with stirring at a temperature between 0° C. to 50° C. until the starting materials have completely disappeared and the mixture is then filtered. Following concentration under vacuum, the amino acid-CD derivative is recrystallized, preferably from water or water-ethanol or methanol.

In another aspect, the present invention provides novel amino acid-CD derivatives, which can be prepared according to methods (i) and (iii) of the invention.

In one embodiment, the amino acid-CD derivative is a mono(6-aminoethylamino-6 deoxy)cyclodextrin covalently linked via the 6-position CD-NH—CH₂—CH₂—NH— group to the functional side group of an α-amino acid selected from aspartic acid, glutamic acid, lysine, tyrosine, cysteine, serine, threonine and histidine. Examples of such derivatives are represented by the compounds herein identified as 10, 11, 14, 15, 18 and 19.

In another embodiment, the amino acid-CD derivative is a mono(6-amino-6 deoxy)cyclodextrin covalently linked via the 6-position CD-NH— group to the functional side group of an α-amino acid selected from aspartic acid, glutamic acid, lysine, tyrosine, cysteine, serine, threonine and histidine, wherein the α-amino or both the α-amino and the α-carboxy groups are protected. Examples of such derivatives are represented by the compounds herein identified as 6, 8, 16, and 17.

These compounds are useful as intermediates for the preparation of the CD-containing polymers of the invention and can also be useful in cosmetic or dermatological compositions or for formation of inclusion complexes with active agents for use in the pharmaceutical and cosmetic industries.

As stated above, the new amino acid-CD derivatives of the invention include the following derivatives: the diprotected glutamic acid-CD derivatives 6, 10; the diprotected aspartic acid-CD derivatives 8, 11; the α-carboxy protected glutamic acid-CD and aspartic acid-CD derivatives 14 and 15, respectively; the α-amino protected glutamic acid-CD derivatives 16, 18; the α-amino protected aspartic acid-CD derivatives 17, 19; and the glutamic acid-CD and aspartic acid-CD derivatives 22 and 23, respectively. These derivatives are depicted in Schemes 8-10 herein.

While reducing the present invention to practice, the inventor found surprisingly that covalent linking of two residues of cyclodextrin to one molecule of an amino acid selected from aspartic acid, glutamic acid and lysine produce a compound with a further ‘pouch’ for encapsulation of active agents. Since these novel compounds have no peptidic bond, they are not affected by protease degradation in the body and can thus form very stable complexes with active agents and such compositions will cross the stomach and the small intestine without degradation.

Thus, a further aspect contemplated by the present invention are derivatives comprising two residues of a CD covalently linked to one molecule of glutamic acid, aspartic acid or lysine, herein identified as “di-CD-amino acid derivative” and such derivatives containing an active ingredient encapsulated therein.

The process for production of such di-CD-amino acid derivatives is depicted in Scheme 11. In one embodiment, a modified CD, e.g. compound 4 is reacted with a N-protected amino acid, e.g., the protected glutamic acid 29, thus obtaining the N-protected di-CD-amino acid derivative 28, and deprotection leads to the di-CD-amino acid derivative 31. In another embodiment, the modified CD compound 5 is reacted with the N-protected glutamic acid 29, thus obtaining the N-protected di-CD-amino acid derivative 30, and deprotection leads to the di-CD-amino acid derivative 32.

In preferred embodiments, the di-CD-amino acid derivatives of the invention are represented by the glutamic acid derivatives 28, 30, 31 and 32.

The invention further relates to the di-CD-amino acid derivatives containing an active agent encapsulated within the cavities of the cyclodextrin residues and within the cavity or pouch formed by the amino acid and the two CD residues. The active agent may be a drug for use in pharmaceutical compositions or an agent for use in cosmetic or dermatological products. These encapsulated products are stable and will not undergo protease degradation. In addition, they do not penetrate into the skin and thus may be useful, for example, for encapsulation of active ingredients of suntan compositions and of preservative agents in cosmetic products.

The di-CD-amino acid derivatives by themselves or with the encapsulated ingredient may be used for all applications as described hereinbefore for the CD-containing polymers.

The invention will now be illustrated by the following non-limiting Examples.

### EXAMPLES

In the Examples herein, the derivatives of the invention and the intermediates will be presented by their respective Arabic numbers in bold according to the following List of Compounds. The formulas of most of the compounds appear in the Schemes 8-10 at the end of the description, just before the References.

### List of Compounds

1. β-cyclodextrin (β-CD or CD)
2. Mono-6-deoxy-6-(p-toluenesulfonyl)-β-cyclodextrin (mono-tosyl-CD)
3. Mono-6-deoxy-6-azido-β-cyclodextrin (mono-azido-CD)
4. Mono-6-deoxy-6-amino-β-cyclodextrin (mono-amino-CD)
5. Mono-6-deoxy-6-(2-aminoethylamino)-β-cyclodextrin (mono-ethyldiamino-CD)
6. Mono-6-deoxy-6-[4-(benzyloxycarbonyl)-4-(tert-butyloxycarbonylamino)butyrylamino]-β-cyclodextrin
7. 4-(benzyloxycarbonyl)-4-(tert-butyloxycarbonylamino) butyric acid (N-Boc-L-glutamic acid-1-benzyl ester)
8. Mono-6-deoxy-6-[3-(benzyloxycarbonyl)-3-(tert-butyloxycarbonylamino)propionylamino]-β-cyclodextrin
9. 3-(benzyloxycarbonyl)-3-(tert-butyloxycarbonylamino) propanoic acid (N-Boc-L-aspartic acid-1-benzyl ester)
10. Mono-6-deoxy-6-[4-(benzyloxycarbonyl)-4-(tert-butyloxycarbonylamino)(butyrylamino ethane)amino]-β-cyclodextrin
11. Mono-6-deoxy-6-[3-(benzyloxycarbonyl)-3-(tert-butyloxycarbonylamino)(propionylamino ethane)amino]-β-cyclodextrin
12. Mono-6-deoxy-6-[4-(benzyloxycarbonyl)-4-amino butyryl amino]-β-cyclodextrin
13. Mono-6-deoxy-6-[3-(benzyloxycarbonyl)-3-amino propionyl amino]-β-cyclodextrin
14. Mono-6-deoxy-6-[4-(benzyloxycarbonyl)-4-amino(butyrylamino ethane)amino]-β-cyclodextrin
15. Mono-6-deoxy-6-[3-(benzyloxycarbonyl)-3-amino(propionylamino ethane)amino]-β-cyclodextrin
16. Mono-6-deoxy-6-[4-carboxy-4-(tert-butyloxycarbonylamino)butyrylamino]-β-cyclodextrin
17. Mono-6-deoxy-6-[3-carboxy-3-(tert-butyloxycarbonylamino)propionylamino]-β-cyclodextrin
18. Mono-6-deoxy-6-[4-carboxy-4-(tert-butyloxycarbonylamino)(butyrylamino ethane)amino]-β-cyclodextrin
19. Mono-6-deoxy-6-[3-carboxy-3-(tert-butyloxycarbonylamino)(propionylamino ethane)amino]-β-cyclodextrin
20. Mono-6-deoxy-6-[4-carboxy-4-amino butyrylamino]-β-cyclodextrin
21. Mono-6-deoxy-6-[3-carboxy-3-amino propionylamino]-β-cyclodextrin
22. Mono-6-deoxy-6-[4-carboxy-4-amino(butyrylamino ethane)amino]-β-cyclodextrin
23. Mono-6-deoxy-6-[3-carboxy-3-amino(propionylamino ethane)amino]-β-cyclodextrin
24. poly[mono-6-deoxy-6-[4-carboxy-4-amino butyrylamino]-β-cyclodextrin]
25. poly[mono-6-deoxy-6-[3-carboxy-3-amino propionylamino]-β-cyclodextrin]
26. poly[mono-6-deoxy-6-[4-carboxy-4-amino(butyrylamino ethane)amino]-β-cyclodextrin]
27. poly[mono-6-deoxy-6-[3-carboxy-3-amino(propionylamino ethane)amino]-β-cyclodextrin]
28. 2-(tert-butyloxycarbonylamino)-N¹,N⁵-bis(6-mono-6-deoxy-β-cyclodextrin)pentanediamide
29. 4-carboxy-4-((tert-butyloxy)carbonyl)aminobutyric acid (N-Boc-L-glutamic acid)
30. 3-(tert-butyloxycarbonylamino)-N¹,N⁶-bis(2-((6-mono-6-deoxy-β-cyclodextrin)amino)ethyl)-2-oxohexanediamide
31. 2-amino-N¹,N⁵-di(6-mono-6-deoxy-β-cyclodextrin)pentanediamide
32. 3-amino-N¹,N⁶-bis(2-((6-mono-6-deoxy-β-cyclodextrin)amino)ethyl)-2-oxohexanediamide

### 33. See Scheme 12.

### 34. See Scheme 12.

### Materials and Methods

Cyclodextrins (Aldrich) were dried (12 h) at 110° C./0.1 mmHg in the presence of P₂O₅. Amino acids were obtained from Aldrich, Sigma or Fluka and were used without further purification. 4,4-Dimethylaminopyridine (DMAP, Aldrich), N,N-dicyclohexylcarbodiimide (DCC, Aldrich), 1-Hydroxybenzotriazole (HOBT, Aldrich) were used without further purification. All reagents were of analytical reagent grade. TLC was performed on silica gel 60 TLC plates and silica gel 60 F₂₅₄ PLC plates (Merck) with EtOAc:2-propanol:NH₄OH_{(aq)}:water (7:7:5:4) or 1-butanol:ethanol:NH₄OH_{(aq)}:H₂O (4:5:6:3). Cyclodextrin derivatives were detected by spraying with 5% v/v concentrated sulfuric acid in ethanol and heating at 150° C. ¹H-NMR and ¹³C-NMR spectra were recorded on an FT-200 MHz spectrophotometer with deuterated dimethyl sulfoxide (DMSO) or deuterated water (D₂O) or deuterated chloroform (CDCl₃) as a solvent; chemical shifts were expressed as δ units (ppm).

### Example 1

### Synthesis of Compound 4

Compound 4 (mono-6-deoxy-6-amino-β-cyclodextrin), was synthesized as shown in Schemes 2 and 8, as follows:

### (i) Synthesis of compound 2 (mono-6-deoxy-6-(p-toluenesulfonyl)-β-cyclodextrin)

A three-liter, three-necked, round-bottomed flask equipped with a mechanical stirring and thermometer was charged with β-cyclodextrin (1) hydrate (50 g, 44 mmol) and a solution of 25 g (625 mmol) of sodium hydroxide in 1.0 liter of water. The solution was stirred at 0-5° C. in an ice-water bath and p-toluenesulfonyl chloride (TsCl) (20 g, 105 mmol) was added in one portion. The reaction mixture was stirred vigorously for 2 h at 0-5° C., and then another portion of TsCl (30 g, 157 mmol) was added and the reaction mixture stirred at this temperature for further 3 h. The reaction mixture was filtered in a fritted glass funnel to separate unreacted TsCl. The filtrate was cooled at 0-5° C. while 10% aqueous hydrochloric acid (HCl, 150 ml) was added. The resulting solution was stored overnight in a refrigerator at 0° C., and then filtered. The product was dried and recrystallized from boiling water. Storage provided 14.0 g (25%) of 2 as a white solid. TLC analysis of 2 performed on silica plates (EtOAc:2-propanol:conc. NH₄OH:water—7:7:5:4) showed one major spot (R_{f}=0.45, R_{f} for β-CD=0.21).

¹H NMR (DMSO-d₆) δ: 2.42 (s, 3H), 3.20-3.67 (m, 40H), 4.16-4.20 (m, 1H), 4.32 (d, 1H, J=9), 4.37-4.39 (m, 1H), 4.45-4.48 (m, 2H), 4.52-4.53 (m, 3H), 4.77 (d, 2H, J=3.4), 4.83-4.84 (m, 5H), 5.64-5.85 (m, 14H), 7.42 (d, 2H, J=8.2), and 7.75 (d, 2H, J=8.2). HPLC (Luna 5u NH₂ 100 A, size 250-4.6 mm, mobile phase 65% acetonitrile—35% H₂O, flow 1.2 ml/min), Rₜ (2)=6.4 min., Rₜ (1)=12.3 min.

### (ii) Synthesis of compound 3 (mono-6-deoxy-6-azido-β-cyclodextrin)

Dry compound 2 obtained in (i) above (30.0 g, 23.3 mmol), was suspended in DMF (45 ml), and the reaction mixture was stirred at 70° C. until the solid component was dissolved (˜10 min). Solid KI (1.92 g, 11.6 mmol) and NaN₃ (15.12 g, 233 mmol) were added and the suspension was stirred at 70° C. for 5 h. The reaction mixture was cooled to room temperature and filtered. The filtrate was added to hot ethanol (500 ml), and the precipitate was filtered and washed three times with boiling ethanol (30 ml). The solid residue was dissolved in water (50 ml) and then poured into ethanol (200 ml). The precipitate was filtered and dried under vacuum yielding 16.2 g of 3 as a white crystalline solid (90% yield). The product was recrystallized from water-acetonitrile (1:1). TLC analysis of 3 performed on silica plates (EtOAc:2-propanol:conc. NH₄OH:water—7:7:5:4) showed one major spot (R_{f}=0.35). ¹H NMR (DMSO-d₆) δ: 3.30-3.65 (m, 42H), 4.45-4.53 (m, 6H), 4.83, 4.88 (two d, 7H), 5.62-5.76 (m, 14H). HPLC (Luna 5u NH₂ 100 A, size 250-4.6 mm, mobile phase 65% acetonitrile—35% H₂O, flow 1.2 ml/min), Rₜ=7.7 min.

### (iii) Synthesis of compound 4 (mono-6-deoxy-6-amino-β-cyclodextrin)

Compound 4 was obtained from 3 by reaction with triphenyl phosphine (Ph₃P) and NH₄OH in DMF, as follows:

Compound 3 obtained in (ii) above, (24.1 g, 21 mmol) and Ph₃P (27.55 g, 105 mmol) were dissolved in DMF (150 ml). The reaction mixture was stirred at 25° C. for 2 h. Concentrated ammonia solution (48.2 ml) was added and a white precipitate was formed. The reaction mixture was maintained at room temperature for 10 h. The precipitate was then removed by filtration and the solvent was evaporated under reduced pressure. The residue was added to ethanol (1500 ml). The solid precipitate was filtered, washed with ether (100 ml) and dried under high vacuum yielding 20.1 g (85% yield) of 4. TLC analysis of 4 performed on silica plates (EtOAc:2-propanol:conc. NH₄OH: water—7:7:5:4) showed one major spot (R_{f}=0.20). ¹H NMR (DMSO-d₆) δ: 3.30-3.65 (m, 42H), 4.44-4.46 (m, 6H), 4.83, 4.89 (two d, 7H), 5.62-5.78 (m, 14H). HPLC (Luna 5u NH₂ 100 A, size 250-4.6 mm, mobile phase 65% acetonitrile—35% H₂O, flow 1.2 ml/min), Rₜ=9.6 min.

### Example 2

### Synthesis of Compound 4

Compound 4 was alternatively synthesized directly from compound 2 by reaction with concentrated NH₄OH solution, as follows:

Compound 2 (3.0 g, 2.33 mmol) was dissolved in 50 ml concentrated NH₄OH solution. The reaction mixture was stirred at 60° C. for 5 h, cooled to room temperature and poured into 500 ml acetone to give a white precipitate. TLC analysis performed on silica plates (EtOAc:2-propanol:conc. NH₄OH:water—7:7:5:4) showed a mixture of product 4 (˜70%) and β-CD 1 (˜30%). The product 4 was recrystallized from ethanol-water (3:7). The ¹H NMR data are as in Example 1.

### Example 3

### Synthesis of Compound 5

Compound 5 (mono-6-deoxy-6-(2-aminoethylamino)-β-cyclodextrin), was prepared by reaction of compound 2 with neat 1,2-diaminoethane, as shown in Scheme 9, as follows:

Dry compound 2 (17.44 g, 13.5 mmol) was dissolved in 1,2-diaminoethane (60 ml). The mixture was stirred at 70° C. for 3 h. Unreacted 1,2-diaminoethane was removed by distillation under reduced pressure and the oily residue was poured into acetone (1500 ml). The solid precipitate was filtered and recrystallized from hot ethanol-water (8:2) yielding 15.1 g (95% yield) of 5 as a white crystalline solid. TLC analysis of 5 performed on silica plates (EtOAc:2-propanol:conc. NH₄OH: water—7:7:5:4) showed one major spot (R_{f}=0.15). ¹H NMR (DMSO-d₆) δ: 1.2 (m, 4H), 3.30-3.65 (m, 42H), 4.44-4.46 (m, 6H), 4.85 (m, 7H), 5.62-5.78 (m, 14H).

### Example 4

### Synthesis of Compound 5

In an alternative route, compound 5 was prepared by reaction of 2 with 1,2-diaminoethane in DMF.

Dry compound 2 (1.7 g, 1.35 mmol) was dissolved in DMF (10 ml). 1,2-Diaminoethane (2 ml) was added and the mixture was stirred at 70° C. for 5 h. Solvent was removed by distillation under reduced pressure and the oily residue was poured into acetone (200 ml). The solid precipitate was filtered and recrystallized from hot ethanol-water (8:2) yielding 1.1 g (69% yield) of 5 as a white crystalline solid. TLC and ¹H NMR data are as in example 3.

### Example 5

### Synthesis of compound 5 (mono-6-deoxy-6-(2-aminoethylamino)-β-cyclodextrin)

Compound 5 was prepared by reacting compound 2 with 1,2-diaminoethane and triethylamine, as shown in Scheme 3 as follows:

Dry compound 2 (3.0 g, 2.32 mmol) was dissolved in 1,2-diaminoethane (10 ml, 8.99 g, 150 mmol). Triethylamine (1.452 g, 14 mmol) was added and the mixture was stirred at 70° C. for 2 h. The reaction mixture was cooled to room temperature, then was poured into acetone (500 ml). The solid precipitate was filtered and recrystallized from hot ethanol-water (8:2) yielding 2.4 g (89% yield) of 5 as a white crystalline solid. TLC and ¹H NMR data are as in Example 3.

### Example 6

### Synthesis of Compound 6 (Glutamic Acid-CD Derivative)

Compound 6, mono-6-deoxy-6-[4-(benzyloxycarbonyl)-4-(tert-butyloxyarbonyl mino)utyrylamino]-β-cyclodextrin, was synthesized by coupling compound 4 with the diprotected glutamic acid N-Boc-L-glutamic acid-1-benzyl ester 7, using DCC and HOBT in DMF, as shown in Schemes 4 and 8, as follows:

Compound 7 (0.337 g, 1.0 mmol), HOBT (0.135 g, 1.0 mmol) and DCC (0.206 g, 1.0 mmol), were dissolved in DMF (5 ml) and stirred at 25° C. for 1 h. Compound 4 (1.134 g, 1.0 mmol) was added and the stirring was continued for 24 h at 25° C. Then, the precipitate was filtered and the DMF was removed by evaporation under reduced pressure. The residue was triturated with hot acetone (100 ml), and the precipitate was filtered and dried under vacuum. The product was recrystallized from hot water yielding 1.25 g (86% yield) of 6 as a white crystalline solid. TLC analysis of 6 performed on silica plates (EtOAc:2-propanol:conc. NH₄OH:water-7:7:5:4) showed one major spot (R_{f}=0.56). ¹H NMR (DMSO-d₆) δ: 1.35 (s, 9H), 1.6-2.2 (m, 4H), 3.30-3.65 (m, 42H), 4.45 (m, 6H), 4.85 (m, 7H), 5.1 (s, 2H), 5.62-5.78 (m, 14H), 7.35 (s, 5H). HPLC (Luna 5u NH₂ 100 A, size 250-4.6 mm, mobile phase 65% acetonitrile—35% H₂O, flow 1.2 ml/min), Rₜ=5.8 min.

### Example 7

### Synthesis of Compound 6

Compound 6 was also prepared by coupling compound 4 with diprotected glutamic acid (7) using DCC and HOBT and zeolite Na—X in DMF, as follows.

Compound 7 (0.337 g, 1.0 mmol), HOBT (0.135 g, 1.0 mmol), and DCC (0.206 g, 1.0 mmol) were dissolved in DMF (5 ml) and stirred at 25° C. for 1 h. Compound 4 (1.134 g, 1.0 mmol) and dry zeolite Na—X (0.5 g) were added and the stirring was continued for 24 h at 25° C. The reaction work-up, isolation of the product and TLC and ¹H NMR data are as described in Example 6 above.

### Example 8

### Synthesis of Compound 6

Compound 6 was prepared by coupling compound 4 with diprotected glutamic acid (7) using DCC and 4-dimethylaminopyridine (DMAP) in DMF.

Compound 7 (0.337 g, 1.0 mmol) and DCC (0.206 g, 1.0 mmol) were dissolved in DMF (5 ml) and stirred at 25° C. for 1 h. Compound 4 (1.134 g, 1.0 mmol) and (DMAP, 0.122 g, 1.0 mmol) were added and the stirring was continued for 24 h at 25° C. The reaction work-up, isolation of the product, and TLC and ¹H NMR data are as described in Example 6.

### Example 9

### Synthesis of Compound 8 (Aspartic Acid-CD Derivative)

The aspartic acid-CD derivative 8 mono-6-deoxy-6-[3-(benzyloxycarbonyl)-3-(tert-butyloxycarbonylamino)propionylamino]-β-cyclodextrin, was synthesized by coupling compound 4 with the N-Boc-L-aspartic acid-1-benzyl ester 9 using DCC and HOBT in DMF, as shown in Schemes 4 and 8.

The preparation of 8 was similar to the preparation of 6 described in Example 6 above, but using 2 (0.323 g, 1.0 mmol) as the diprotected amino acid. The crude precipitate obtained in the reaction was recrystallized from hot water yielding 1.35 g (93% yield) of 8 as a white crystalline solid. TLC analysis of 8 performed on silica plates (EtOAc:2-propanol:conc. NH₄OH:water—7:7:5:4) showed one major spot (R_{f}=0.44). ¹H NMR (DMSO-d₆) δ: 1.35 (s, 9H), 3.30-3.65 (m, 42H), 4.45 (m, 6H), 4.85 (m, 7H), 5.1 (s, 2H), 5.62-5.78 (m, 14H), 7.35 (s, 5H). HPLC (Luna 5u NH₂ 100 A, size 250-4.6 mm, mobile phase 65% acetonitrile—35% H₂O, flow 1.2 ml/min), Rₜ=10.1 min.

### Example 10

### Synthesis of Compound 8 (Aspartic Acid-CD Derivative)

The preparation of compound 8 by coupling compound 4 with diprotected aspartic acid using DCC and HOBT and zeolite Na—X in DMF was carried out as described for compound 6 in Example 7, but using compound 2 (0.323 g, 1.0 mmol) as the diprotected amino acid. The reaction work-up, isolation of the product and TLC and ¹H NMR data are as described in Example 9.

### Example 11

### Synthesis of Compound 8

The preparation of compound 8 by coupling compound 4 with diprotected aspartic acid using DCC and DMAP in DMF was carried out as described for compound 6 in Example 8, but using compound 9 (0.323 g, 1.0 mmol) as the diprotected amino acid. The reaction work-up, isolation of the product and TLC and ¹H NMR data are as described in Example 9.

### Example 12

### Synthesis of Compound 10 (Ethylenediamino-Glutamic Acid-CD Derivative)

Cyclodextrin derivative 10 mono-6-deoxy-6-[4-(benzyloxycarbonyl)-4-(tert-butyloxycarbonylamino)(butyrylamino ethane)amino]-β-cyclodextrin, was synthesized by coupling compound 5 with the diprotected glutamic acid N-Boc-L-glutamic acid-1-benzyl ester (7), using DCC and HOBT in DMF as shown in Schemes 4 and 9, as follows:

Compound 7 (0.337 g, 1.0 mmol), HOBT (0.135 g, 1.0 mmol), and DCC (0.206 g, 1.0 mmol) were dissolved in DMF (5 ml) and stirred at 25° C. for 1 h. Compound 5 (1.177 g, 1.0 mmol) was added and the stirring was continued for 24 h at 25° C. The precipitate was filtered and the DMF was removed by evaporation under reduced pressure. The residue was triturated with hot acetone (100 ml), and the precipitate was filtered and dried under vacuum. The product was recrystallized from hot water yielding 1.1 g (73% yield) of 10 as a white crystalline solid. TLC analysis of 10 performed on silica plates (EtOAc:2-propanol:conc. NH₄OH:water—7:7:5:4) showed one major spot (R_{f}=0.55). ¹H NMR (DMSO-d₆) δ: 1.35 (s, 9H), 1.6-2.2 (m, 8H), 3.30-3.65 (m, 42H), 4.45 (m, 6H), 4.85 (m, 7H), 5.1 (s, 2H), 5.62-5.78 (m, 14H), 7.35 (s, 5H).

### Example 13

### Synthesis of Ethylenediamino-Aspartic Acid-CD Derivative (Compound 11)

The preparation of derivative 11 mono-6-deoxy-6-[3-(benzyloxycarbonyl)-3-(tert-butyloxycarbonylamino)(propionylamino ethane)amino]-β-cyclodextrin, was carried out as described in Example 12 above, but using the diprotected aspartic acid 9 (0.323 g, 1.0 mmol) for coupling with compound (5), using DCC and HOBT in DMF (see Scheme 9).

The crude precipitate obtained in the reaction was recrystallized from hot water yielding 1.05 g (71% yield) of 11 as a white crystalline solid. TLC analysis of 11 performed on silica plates (EtOAc:2-propanol:conc. NH₄OH:water—7:7:5:4) showed one major spot (R_{f}=0.41). ¹H NMR (DMSO-d₆) δ: 1.35 (s, 9H), 1.6 (m, 4H), 3.30-3.65 (m, 42H), 4.45 (m, 6H), 4.85 (m, 7H), 5.1 (s, 2H), 5.62-5.78 (m, 14H), 7.35 (s, 5H).

### Example 14

### Synthesis of Compound 12

Compound 12 mono-6-deoxy-6-[4-(benzyloxycarbonyl)-4-amino butyryl amino]-β-cyclodextrin was obtained by removing the N-protecting Boc group from compound 6 using CF₃COOH in CH₂Cl₂ (Scheme 8).

Compound 6 (1.453 g, 1.0 mmol) was dissolved in TFA (5 ml) and CH₂Cl₂ (5 ml), and the mixture was stirred at 25° C. for 3 h. The solvent was removed by evaporation under reduced pressure (<25° C.). The residue was dissolved in water (5 ml) and poured into methanol (200 ml). The white precipitate was filtered and dried under vacuum (93% yield). TLC analysis of 12 performed on silica plates (EtOAc:2-propanol:conc. NH₄OH:water—7:7:5:4) showed one major spot (R_{f}=0.37). ¹H NMR (D₂O) δ: 1.8-2.2 (m, 4H), 3.47-3.84 (m, 42H), 4.9-5.1 (m, 9H), 7.23-7.42 (m, 5H). ¹H NMR (DMSO-d₆) δ: 1.8-2.2 (m, 4H), 3.30-3.65 (m, 42H), 4.45 (m, 6H), 4.90 (m, 7H), 5.18 (s, 2H), 5.76-5.82 (m, 14H), 7.45 (s, 5H).

### Example 15

### Synthesis of Compound 13

Compound 13 mono-6-deoxy-6-[3-(benzyloxycarbonyl)-3-amino propionyl amino]-β-cyclodextrin, was obtained by removing the N-protecting Boc group from compound 8 using CF₃COOH in CH₂Cl₂ (Scheme 8).

Compound 8 (1.439 g, 1.0 mmol) was dissolved in TFA (5 ml) and CH₂Cl₂ (5 ml) and the mixture was treated as described in Example 14 above, to yield a white precipitate that was filtered and dried under vacuum (91% yield). TLC analysis of 13 performed on silica plates (EtOAc:2-propanol:conc. NH₄OH:water—7:7:5:4) showed one major spot (R_{f}=0.22). ¹H NMR (D₂O) δ: 1.8-2.2 (m, 2H), 3.47-3.84 (m, 42H), 4.9-5.1 (m, 9H), 7.23-7.42 (m, 5H). ¹H NMR (DMSO-d₆) δ: 1.8-2.2 (m, 2H), 3.30-3.65 (m, 42H), 4.45 (m, 6H), 4.90 (m, 7H), 5.18 (s, 2H), 5.76-5.82 (m, 14H), 7.45 (s, 5H).

### Example 16

### Synthesis of Compound 14

Compound 14 mono-6-deoxy-6-[4-(benzyloxycarbonyl)-4-amino(butyrylamino ethane)amino]-β-cyclodextrin, was obtained by removing the N-protecting Boc group from compound 10 using CF₃COOH in CH₂Cl₂ (Scheme 9).

Compound 10 (1.496 g, 1.0 mmol) was dissolved in TFA (5 ml) and (CH₂Cl₂ (5 ml) and the mixture was treated as described in Example 14 to yield a white precipitate that was filtered and dried under vacuum (87% yield). TLC analysis of 14 performed on silica plates (EtOAc:2-propanol:conc. NH₄OH:water—7:7:5:4) showed one major spot (R_{f}=0.31). ¹H NMR (D₂O) δ: 1.8-2.2 (m, 8H), 3.47-3.84 (m, 42H), 4.9-5.1 (m, 9H), 7.23-7.42 (m, 5H).

### Example 17

### Synthesis of Compound 15

Compound 15 mono-6-deoxy-6-[3-(benzyloxycarbonyl)-3-amino(propionyl-amino-ethane)amino]-β-cyclodextrin, was obtained by removing the N-protecting Boc group from the compound 11 using CF₃COOH in CH₂Cl₂ (Scheme 9).

Compound 11 (1.482 g, 1.0 mmol) was dissolved TFA and CH₂Cl₂ and the mixture was treated as described in Example 14 to yield a white precipitate that was filtered and dried under vacuum (83% yield). TLC analysis of 15 performed on silica plates (EtOAc:2-propanol:conc. NH₄OH:water—7:7:5:4) showed one major spot (R_{f}=0.19). ¹H NMR (D₂O) δ: 1.8-2.2 (m, 6H), 3.47-3.84 (m, 42H), 4.9-5.1 (m, 9H), 7.23-7.42 (m, 5H).

### Example 18

### Synthesis of Compound 16

Compound 16 mono-6-deoxy-6-[4-carboxy-4-(tert-butyloxycarbonylamino) butyrylamino]-β-cyclodextrin, was obtained by removing the protecting benzyl group from compound 6 using aqueous NaOH, as shown in Scheme 8, as follows:

Compound 6 (1.453 g, 1.0 mmol) was dissolved in 1M NaOH solution (20 ml) and the mixture was stirred at 25° C. for 5 h. The solvent was removed by evaporation under reduced pressure (<25° C.). The residue was poured into methanol (200 ml). The white precipitate was filtered and dried under vacuum (92% yield). TLC analysis of 16 performed on silica plates (EtOAc:2-propanol:conc. NH₄OH:water—7:7:5:4) showed one major spot (R_{f}=0.27). ¹H NMR (D₂O) δ: 1.41 (s, 9H), 1.6-2.8 (m, 4H), 3.43-3.93 (m, 42H), 4.93-4.95 (m, 7H).

### Example 19

### Synthesis of Compound 17

Compound 17 mono-6-deoxy-6-[3-carboxy-3-(tert-butyloxycarbonylamino) propionylamino]-β-cyclodextrin, was obtained by removing the protecting benzyl group from compound 8 using aqueous NaOH (Scheme 8).

Compound 8 (1.439 g, 1.0 mmol) was dissolved in 1M NaOH solution (20 ml) and the mixture was treated as described in Example 18 to yield a white precipitate that was filtered and dried under vacuum (88% yield). TLC analysis of 17 performed on silica plates (EtOAc:2-propanol:conc. NH₄OH:water—7:7:5:4) showed one major spot (R_{f}=0.16). ¹H NMR (D₂O) δ: 1.41 (s, 9H), 1.6-2.8 (m, 2H), 3.43-3.93 (m, 42H), 4.93-4.95 (m, 7H).

### Example 20

### Synthesis of Compound 18

Compound 18 mono-6-deoxy-6-[4-carboxy-4-(tert-butyloxycarbonylamino) (butyrylamino ethane)amino]-β-cyclodextrin, was obtained by removing the protecting benzyl group from compound 10 using aqueous NaOH (Scheme 9).

Compound 10 (1.496 g, 1.0 mmol) was dissolved in 1M NaOH solution (20 ml) and treated as described in Example 18 to yield a white precipitate that was filtered and dried under vacuum (81% yield). TLC analysis of 18 performed on silica plates (EtOAc:2-propanol:conc. NH₄OH:water—7:7:5:4) showed one major spot (R_{f}=0.23). ¹H NMR (D₂O) δ: 1.41 (s, 9H), 1.6-2.8 (m, 8H), 3.43-3.93 (m, 42H), 4.93-4.95 (m, 7H).

### Example 21

### Synthesis of Compound 19

Compound 19 mono-6-deoxy-6-[3-carboxy-3-(tert-butyloxycarbonylamino) (propionylamino ethane)amino]-β-cyclodextrin, was obtained by removing the protecting benzyl group from compound 11 using aqueous NaOH (Scheme 9).

Compound 11 (1.482 g, 1.0 mmol) was dissolved in 1M NaOH solution (20 ml) and treated as described in Example 18 to yield a white precipitate that was filtered and dried under vacuum (85% yield). TLC analysis of 19 performed on silica plates (EtOAc:2-propanol:conc. NH₄OH:water—7:7:5:4) showed one major spot (R_{f}=0.15). ¹H NMR (D₂O) δ: 1.41 (s, 9H), 1.6-2.8 (m, 6H), 3.43-3.93 (m, 42H), 4.93-4.95 (m, 7H).

### Example 22

### Synthesis of Compound 16

Compound 16 was obtained by removing the protecting benzyl group from compound 6 using aqueous NH₄OH (Scheme 8), as follows:

Compound 6 (1.453 g, 1.0 mmol) was dissolved in concentrated NH₄OH solution (50 ml) and the mixture was stirred at 25° C. for 24 h. The solvent was removed by evaporation under reduced pressure (<25° C.). The residue was poured into methanol (200 ml). The white precipitate was filtered and dried under vacuum. The TLC and NMR data are as in Example 18.

### Example 23

### Synthesis of Compound 17

Compound 17 was obtained by removing the protecting benzyl group from the compound 8 using aqueous NH₄OH, as described in Example 22 above (and shown in Scheme 9), and the TLC and NMR data are as in Example 19.

### Example 24

### Synthesis of Compound 18

Compound 18 was obtained by removing the protecting benzyl group from the compound 10 using aqueous NH₄OH, as described in Example 22 above (and shown in Scheme 9), and the TLC and NMR data are as in Example 20.

### Example 25

### Synthesis of Compound 19

Compound 19 was obtained by removing the protecting benzyl group from the compound 11 using aqueous NH₄OH, as described in Example 22 above (and shown in Scheme 9), and the TLC and NMR data are as in Example 21.

### Example 26

### Synthesis of Compound 20

Compound 20 mono-6-deoxy-6-[4-carboxy-4-amino butyrylamino]-β-cyclodextrin, was obtained by removing the N-protecting Boc group and_benzyl group from compound 6 as shown in Scheme 10, as follows:

Compound 6 (1.453 g, 1.0 mmol) was dissolved in TFA (5 ml) and (CH₂Cl₂ (5 ml) and the mixture was stirred at 25° C. for 3 h. The solvent was removed by evaporation under reduced pressure (<25° C.). The residue was dissolved in 1M NaOH (20 ml) and the mixture was stirred at 25° C. for 5 h. The solvent was removed by evaporation under reduced pressure (<25° C.) and the residue was poured into methanol (200 ml). The white precipitate was filtered and dried under vacuum (65% yield). TLC analysis of 20 performed on silica plates (EtOAc:2-propanol:conc. NH₄OH:water—7:7:5:4) showed one major spot (R_{f} 0.20). ¹H NMR (D₂O) δ: 1.8-2.2 (m, 4H), 3.47-3.84 (m, 42H), 4.9-5.1 (m, 7H).

### Example 27

### Synthesis of Compound 21

Compound 21 mono-6-deoxy-6-[3-carboxy-3-amino propionylamino]-β-cyclodextrin, was obtained by removing the N-protecting Boc group and the benzyl group from compound 8 (Scheme 10).

Compound 8 (1.439 g, 1.0 mmol) was treated with TFA and CH₂Cl₂ to remove the N-protecting Boc group and with NaOH to remove the benzyl group as described above in Example 26, to yield a white precipitate that was filtered and dried under vacuum (72% yield). TLC analysis of 21 performed on silica plates (EtOAc:2-propanol:conc. NH₄OH:water—7:7:5:4) showed one major spot (R_{f}=0.13). ¹H NMR (D₂O) δ: 1.8-2.2 (m, 2H), 3.47-3.84 (m, 42H), 4.9-5.1 (m, 7H).

### Example 28

### Synthesis of Compound 22

Compound 22 mono-6-deoxy-6-[4-carboxy-4-amino(butyrylamino ethane) amino]-β-cyclodextrin, was obtained by removing the N-protecting Boc group and the benzyl group from compound 10 (Scheme 10).

Compound 10 (1.496 g, 1.0 mmol) was treated with TFA and CH₂Cl₂ to remove the N-protecting Boc group and with NaOH to remove the benzyl group as described above in Example 26, to yield a white precipitate that was filtered and dried under vacuum (55% yield). TLC analysis of 22 performed on silica plates (EtOAc:2-propanol:conc. NH₄OH:water—7:7:5:4) showed one major spot (R_{f}=0.18). ¹H NMR (D₂O) δ: 1.8-2.2 (m, 8H), 3.47-3.84 (m, 42H), 4.9-5.1 (m, 7H).

### Example 29

### Synthesis of Compound 23

Compound 23 mono-6-deoxy-6-[3-carboxy-3-amino(propionylamino ethane) amino]-β-cyclodextrin, was obtained by removing the N-protecting Boc group and the benzyl group from compound 11 (Scheme 10).

Compound 11 (1.482 g, 1.0 mmol) was treated with TFA and CH₂Cl₂ to remove the N-protecting Boc group and with NaOH to remove the benzyl group as described above in Example 26, to yield a white precipitate that was filtered and dried under vacuum (63% yield). TLC analysis of 23 performed on silica plates (EtOAc:2-propanol:conc. NH₄OH:water—7:7:5:4) showed one major spot (R_{f}=0.11). ¹H NMR (D₂O) δ: 1.8-2.2 (m, 6H), 3.47-3.84 (m, 42H), 4.9-5.1 (m, 7H).

### Example 30

### Synthesis of Homo-Polymer 24

Homo-polymer 24 was obtained by coupling of compound 20 using DCC and HOBT in DMF, as shown in Scheme 10.

Compound 20 (1.263 g, 1.0 mmol), HOBT (0.135 g, 1.0 mmol), and DCC (0.206 g, 1.0 mmol) were dissolved in DMF (5 ml) and stirred at 25° C. for 7 days. The precipitate was filtered and the DMF was removed by evaporation under reduced pressure. The residue was dissolved in water (5 ml) and was poured into methanol (300 ml). The precipitate was filtered and dried under vacuum (92% yield) to give a mixture of polypeptides containing cyclodextrins. TLC analysis of 24 performed on silica plates (1-butanol:ethanol:NH₄OH:H₂O—4:5:3:5) showed a mixture of five polypeptides (R_{f}=0.06; 0.11; 0.17; 0.26; 0.34). ¹H NMR (D₂O) δ: 1.8-2.2 (m, 4H), 3.47-3.84 (m, 42H), 4.9-5.1 (m, 7H).

### Example 31

### Synthesis of Homopolymer 25

Homopolymer 25 was obtained by coupling of compound 21 (1.249 g, 1.0 mmol), using DCC and HOBT in DMF (Scheme 10), as described for homo-polymer 24 in Example 30 above. The obtained precipitate was filtered and dried under vacuum (85% yield) to give a mixture of polypeptides containing cyclodextrins. TLC analysis of 25 performed on silica plates (1-butanol:ethanol:NH₄OH:H₂O—4:5:3:5) showed a mixture of polypeptides (R_{f}=0.02-0.29). ¹H NMR (D₂O) δ: 1.8-2.2 (m, 2H), 3.47-3.84 (m, 42H), 4.9-5.1 (m, 7H).

### Example 32

### Synthesis of Homopolymer 26

Homopolymer 26 was obtained by coupling of compound 22 (1.307 g, 1.0 mmol), using DCC and HOBT in DMF (Scheme 10), as described for homo-polymer 24 in Example 30 above. The obtained precipitate was filtered and dried under vacuum (74% yield) to give a mixture of polypeptides containing cyclodextrins. TLC analysis of 26 performed on silica plates (1-butanol:ethanol:NH₄OH:H₂O—4:5:3:5) showed a mixture of polypeptides (R_{f}=0.05-0.33). ¹H NMR (D₂O) δ: 1.8-2.2 (m, 8H), 3.47-3.84 (m, 42H), 4.9-5.1 (m, 7H).

### Example 33

### Synthesis of Homopolymer 27

Homopolymer 27 was obtained by coupling of compound 23 (1.293 g, 1.0 mmol), using DCC and HOBT in DMF (Scheme 10), as described for homo-polymer 24 in Example 30 above. The obtained precipitate was filtered and dried under vacuum (79% yield) to give a mixture of polypeptides containing cyclodextrins. TLC analysis of 27 performed on silica plates (1-butanol:ethanol:NH₄OH:H₂O—4:5:3:5) showed a mixture of polypeptides (R_{f}=0.01-0.23). ¹H NMR (D₂O) δ: 1.8-2.2 (m, 6H), 3.47-3.84 (m, 42H), 4.9-5.1 (m, 7H).

### Example 34

### Synthesis of Di-CD-Amino Acid Derivative 28

Di-CD-glutamic acid derivative 28 2-(tert-butyloxycarbonylamino)-N¹,N⁵-bis(6-mono-6-deoxy-β-cyclodextrin) pentanediamide, was obtained by di-coupling one molecule of N-protected glutamic acid 29 (N-Boc-L-glutamic acid) with two moieties of compound 4 (mono-6-deoxy-6-amino-β-cyclodextrin), using DCC and HOBT in DMF (mono amino-CD:amino acid 2:1), as shown in Scheme 11, as follows:

N-protected amino acid 29 (0.247 g, 1.0 mmol), HOBT (0.270 g, 2.0 mmol), and DCC (0.412 g, 2.0 mmol) were dissolved in DMF (10 ml) and stirred at 25° C. for 1 h. Compound 4 (2.268 g, 2.0 mmol) was added and the stirring was continued for 48 h at 25° C. The precipitate was filtered and the DMF was removed by evaporation under reduced pressure. The residue was triturated with hot acetone (100 ml). The precipitate was filtered and dried under vacuum. Derivative 28 was obtained as a white solid (77% yield). TLC analysis of 28 performed on silica plates (1-butanol:ethanol:NH₄OH:water—4:5:6:2) showed one major spot (R_{f}=0.16). ¹H NMR (DMSO-d₆) δ: 1.38 (s, 9H), 1.71-2.15 (m, 4H), 3.16-3.49 (m, 84H), 4.30-4.44 (m, 12H), 4.68 (m, 14H), 5.62-5.78 (m, 28H). HPLC (Luna 5u NH₂ 100 A, size 250-4.6 mm, mobile phase 65% acetonitrile—35% H₂O, flow 1.2 ml/min), Rₜ=44.6 min.

### Example 35

### Synthesis of 30

Di-CD-glutamic acid 30 3-(tert-butyloxycarbonylamino)-N¹,N⁶-bis(2-((6-mono-6-deoxy-β-cyclodextrin)amino)ethyl)-2-oxohexanediamide, was obtained by di-coupling one molecule of N-protected glutamic acid 29 with two moieties of compound 5 (mono-6-deoxy-6-(2-aminoethylamino)-β-cyclodextrin, 2.354 g, 2.0 mmol), using DCC and HOBT in DMF (mono amino-CD:amino acid 2:1) (Scheme 11), as described for dimer 28 in Example 34 above. Dimer 30 was obtained as a white solid (77% yield). TLC analysis of 30 performed on silica plates (1-butanol:ethanol:NH₄OH:water—4:5:6:2) showed one major spot (R_{f}=0.12). ¹H NMR (DMSO-d₆) δ: 1.38 (s, 9H), 1.71-2.15 (m, 12H), 3.21-3.73 (m, 84H), 4.42-4.46 (m, 12H), 4.80-4.82 (m, 14H), 5.68-5.77 (m, 28H). HPLC (Luna 5u NH₂ 100 A, size 250-4.6 mm, mobile phase 65% acetonitrile—35% H₂O, flow 1.2 ml/min), Rₜ=33.5 min.

### Example 36

### Synthesis of 31

Di-CD-glutamic acid derivative 31 2-amino-N¹,N⁵-di(6-mono-6-deoxy-β-cyclodextrin) pentanediamide, was obtained by removing the N-protecting Boc group from 28 using TFA in CH₂Cl₂, as shown in Scheme 11, as follows:

28 (0.248 g, 0.1 mmol) was dissolved in TFA (2 ml) and (CH₂Cl₂ (2 ml) and the mixture was stirred at 25° C. for 3 h. The solvent was removed by evaporation under reduced pressure (<25° C.). The residue was dissolved in water (1 ml) and poured into acetone (100 ml). The white precipitate was filtered and dried under vacuum (84% yield). TLC analysis of 31 performed on silica plates (1-butanol:ethanol:NH₄OH:water—4:5:6:3) showed one major spot (R_{f}=0.15). ¹H NMR (DMSO-d₆) δ: 1.71-2.15 (m, 4H), 3.16-3.49 (m, 84H), 4.30-4.44 (m, 12H), 4.68 (m, 14H), 5.62-5.78 (m, 28H).

### Example 37

### Synthesis of 32

The compound 32 3-amino-N¹,N⁶-bis(2-((6-mono-6-deoxy-β-cyclodextrin) amino)ethyl)-2-oxohexanediamide, was obtained by removing the N-protecting Boc group from 30 (0.257 g, 0.1 mmol) using TFA in CH₂Cl₂ (Scheme 11), as described for 31 in Example 36 above. Compound 32 was obtained as a white precipitate, filtered and dried under vacuum (84% yield). TLC analysis of 32 performed on silica plates (1-butanol:ethanol:NH₄OH:water—4:5:6:3) showed one major spot (R_{f}=0.11). ¹H NMR (DMSO-d₆) δ: 1.71-2.15 (m, 12H), 3.16-3.49 (m, 84H), 4.30-4.44 (m, 12H), 4.68 (m, 14H), 5.62-5.78 (m, 28H).

### Example 38

### Synthesis of 33

Compound 33 was obtained by coupling of compound 12 with compound 16 using HOBT and DCC in DMF, as shown in Scheme 12, as follows:

Compound 12 (1.365 g, 1.0 mmol), 16 (1.363 g, 1.0 mmol), HOBT (0.270 g, 2.0 mmol), and DCC (0.412 g, 2.0 mmol) were dissolved in DMF (10 ml) and stirred at 25° C. for 7 days. The precipitate was filtered and the DMF was removed by evaporation under reduced pressure. The residue was triturated with hot acetone (100 ml). The precipitate was filtered and dried under vacuum (65% yield). TLC analysis of 33 performed on silica plates (EtOAc:2-propanol:conc. NH₄OH:water—7:7:5:4) showed one major spot (R_{f}=0.12). ¹H NMR (DMSO-d₆) δ: 1.35 (s, 9H), 1.6-2.2 (m, 8H), 3.30-3.65 (m, 84H), 4.45 (m, 12H), 4.85 (m, 14H), 5.1 (s, 2H), 5.62-5.78 (m, 28H), 7.35 (s, 5H).

### Example 39

### Synthesis of 34

Compound 34 was obtained by removing the N-protecting Boc group and the benzyl group from compound 33 using TFA and NaOH, as shown in Scheme 12.

Compound 33 (2.710 g, 1.0 mmol) was dissolved in TFA (10 ml) and CH₂Cl₂ (10 ml) and the mixture was stirred at 25° C. for 5 h. The solvent was removed by evaporation under reduced pressure (<25° C.). The residue was dissolved in 1M NaOH (50 ml) and the mixture was stirred at 25° C. for 12 h. The solvent was removed by evaporation under reduced pressure (<25° C.) and the residue was poured into acetone (200 ml). The white precipitate was filtered and dried under vacuum (77% yield). TLC analysis of 34 performed on silica plates (EtOAc:2-propanol:conc. NH₄OH:water—7:7:5:4) showed one major spot (R_{f}=0.05). ¹H NMR (DMSO-d₆) δ: 1.6-2.2 (m, 8H), 3.30-3.65 (m, 84H), 4.45 (m, 12H), 4.85 (m, 14H), 5.62-5.78 (m, 28H).

### Example 40

### Preparation of CD-Containing Peptides by Grafting Modified Cyclodextrin onto Peptides

A general procedure for the grafting of mono amino-CDs onto a peptide having an amino acid residue with a COOH functional side group is depicted in Scheme 13. For the preparation of a CD-containing peptide, a N-Boc-peptide (e.g. compound 36), HOBT, and DCC are dissolved in DMF and stirred at 25° C. for 1 h. A modified CD, e.g., compound 4 or 5, is added and the stirring is continued for 48 h at 25° C. The precipitate is filtered and the DMF is removed by evaporation under reduced pressure. The residue is triturated with hot methanol. The precipitate is filtered and dried under vacuum to obtain the desired CD-containing polypeptide.

### Example 41

### General Procedure for Encapsulation of Guest Molecules by Dipeptide 34

For the encapsulation process, a guest molecule (e.g., thymol, vitamin E) (0.03 mmol) and dipeptide 34 (0.01 mmol) are completely dissolved in a mixed solution of ethanol and water (10%:90%) and stirred for 3 days at room temperature. After evaporating the ethanol from the stirred solution, the uncomplexed guest molecule is removed by filtration. The filtrate is again evaporated to remove water and dried in vacuum to give dipeptide 34-encapsulated guest complex (yield 90%).

### Example 42

### General Procedure of Coupling Diprotected Amino Acid with Native cyclodextrin

Diprotected amino acid (glutamic acid or aspartic acid, 1.0 mmol), HOBT (1.0 mmol), DMAP (1.0 mmol), zeolite (1 g) and DCC (1.0 mmol), are added to DMF (10 ml) and stirred at 25° C. for 2 h. Native cyclodextrin (α-CD or β-CD or γ-CD) (2.0 mmol) is added and the stirring is continued for 2-7 days at 25° C. Then, the solid precipitate is filtered and the DMF is removed by evaporation under reduced pressure. The residue is dissolved in water and purified by reversed-phase chromatography (eluent: 5% acetonitrile/95% water). The product is recrystallized from hot water (50-60% yield).

In the following pages, the Schemes 1-13 mentioned above are depicted. In the schemes, n in the cyclodextrin ring means a value of 6, 7 or 8.

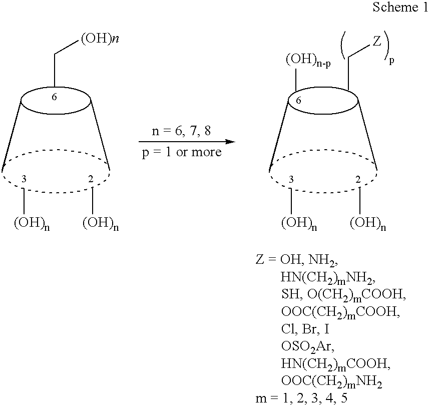

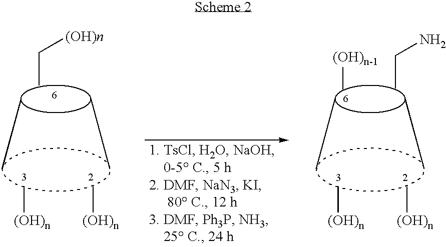

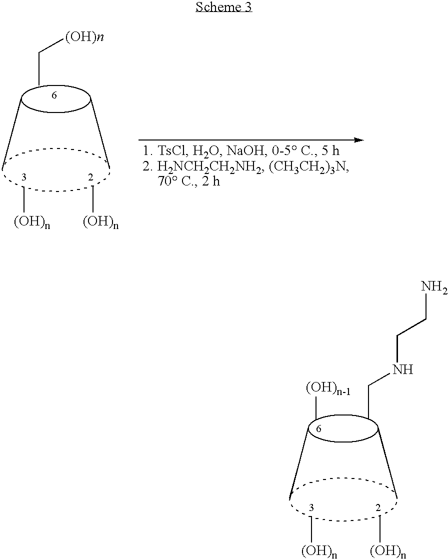

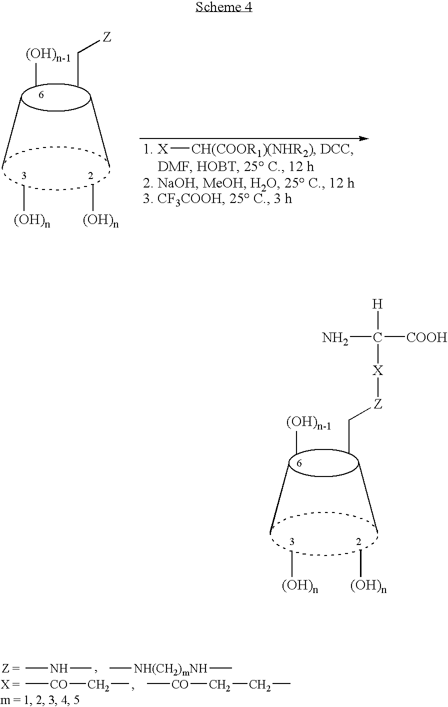

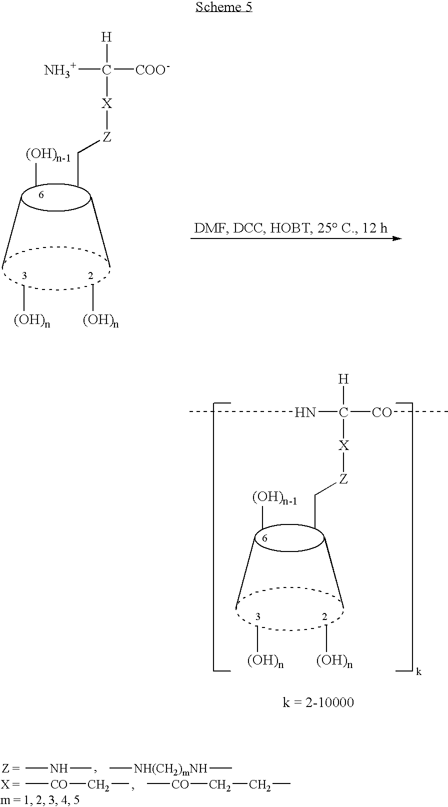

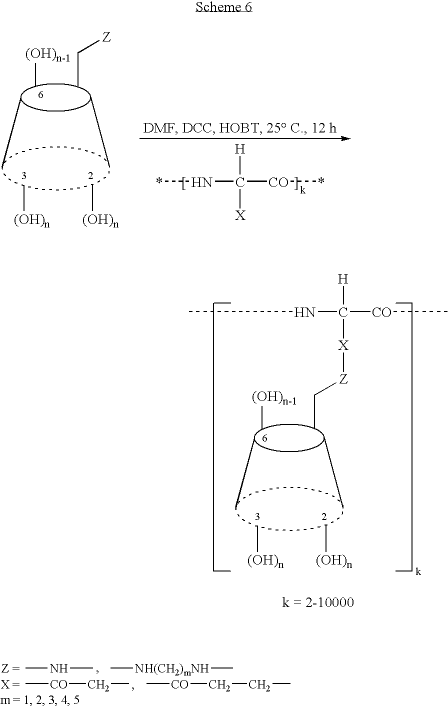

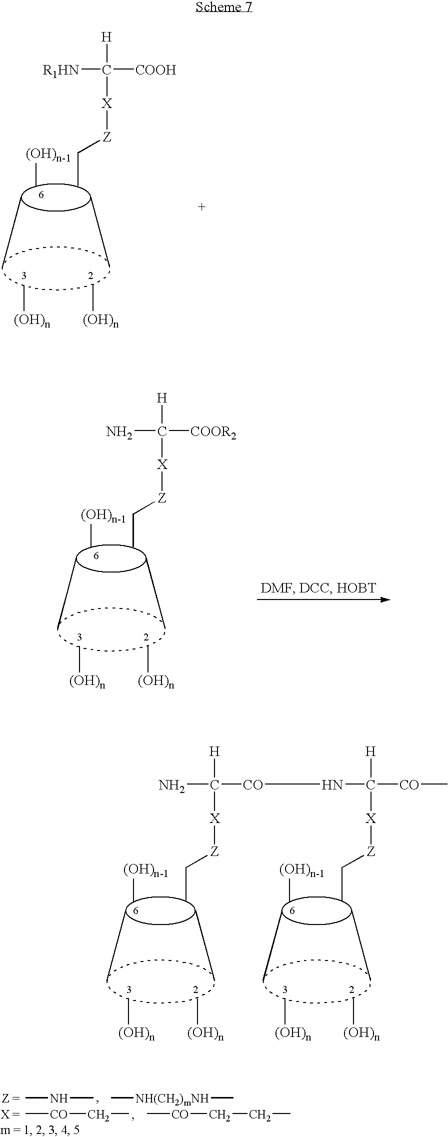

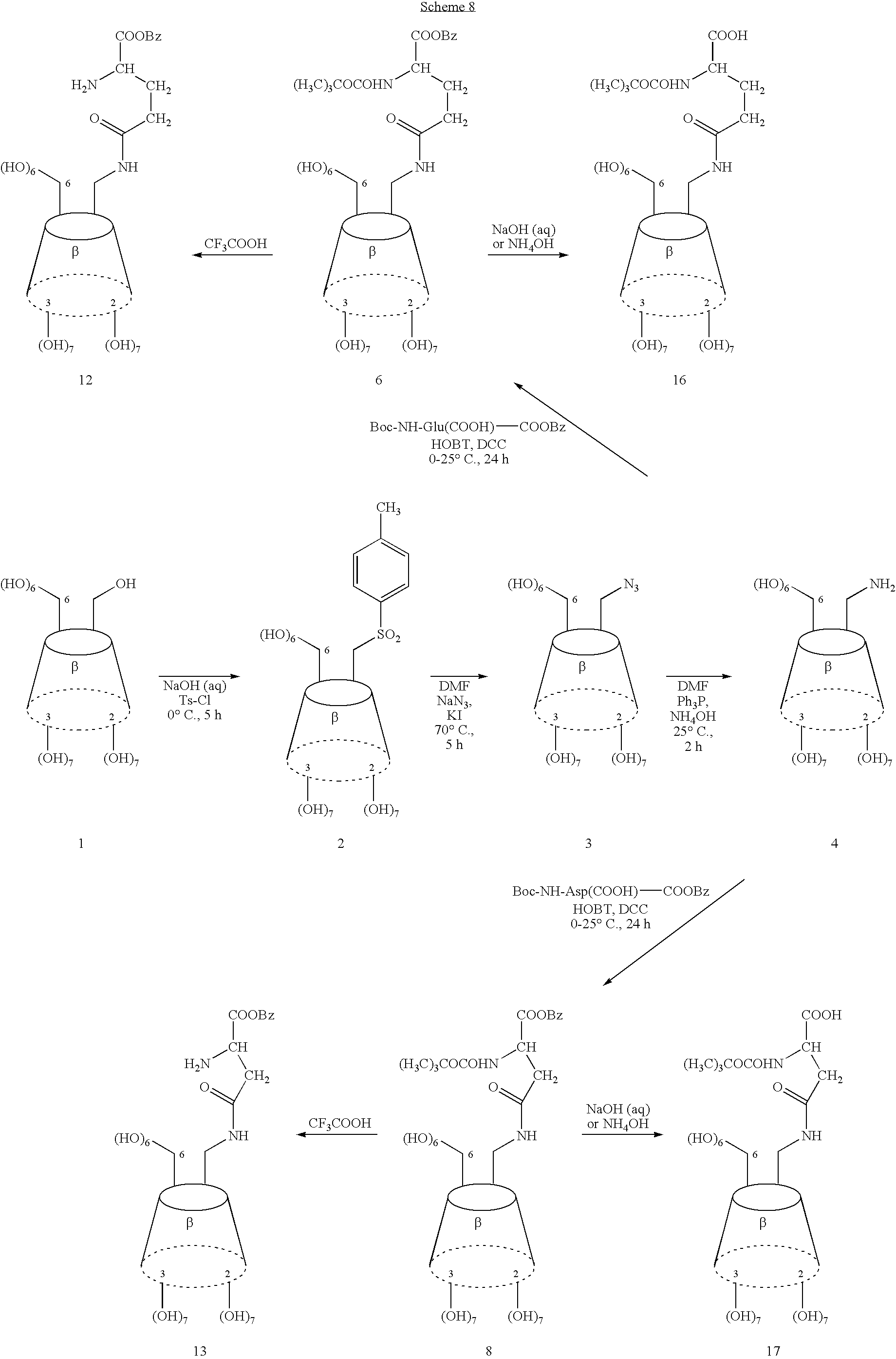

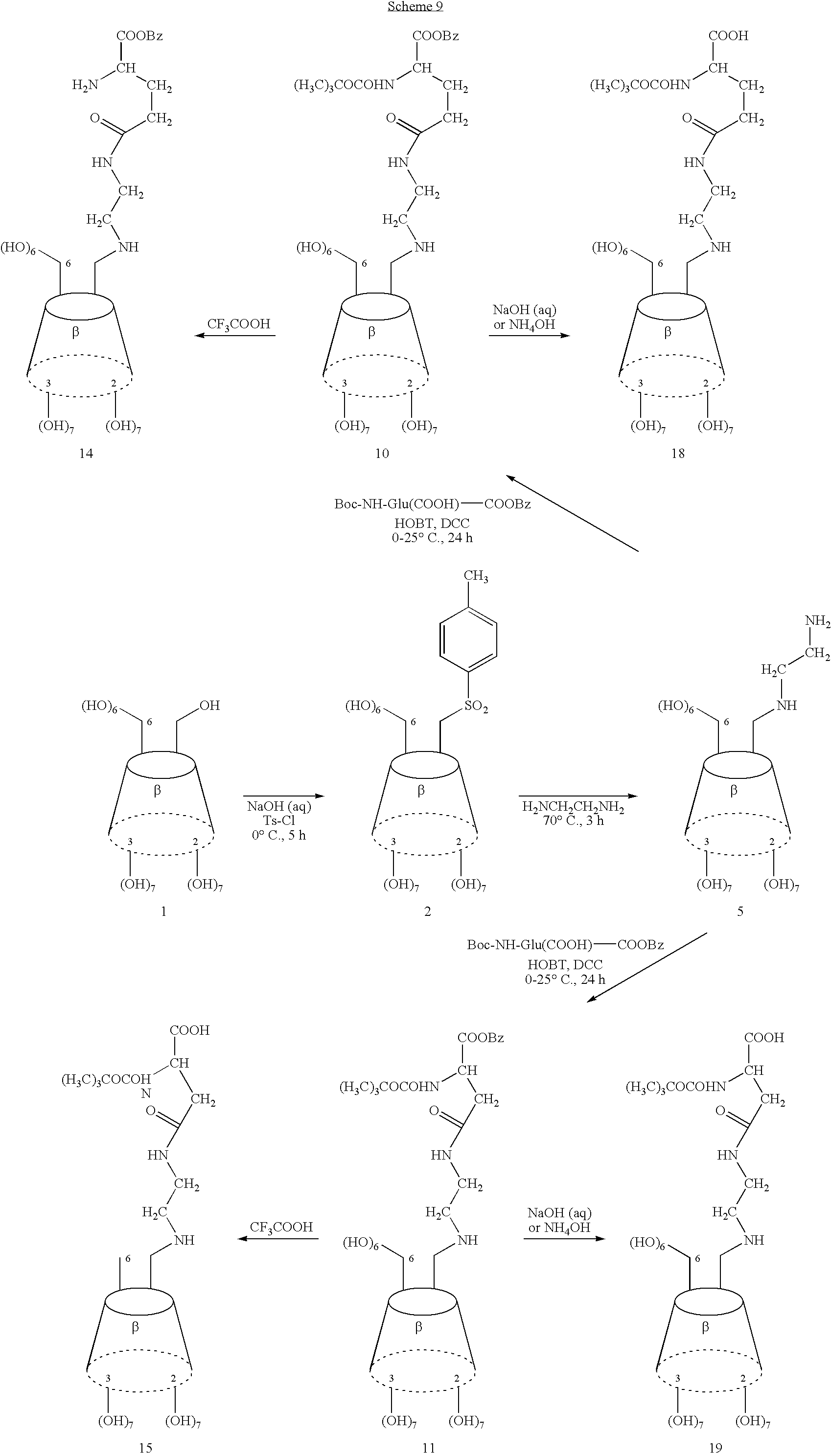

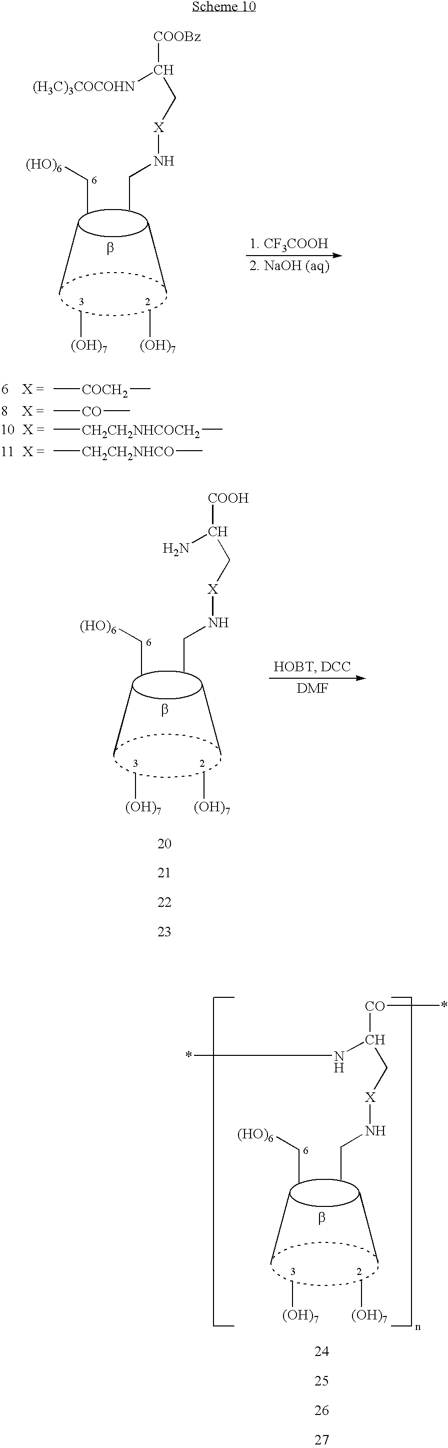

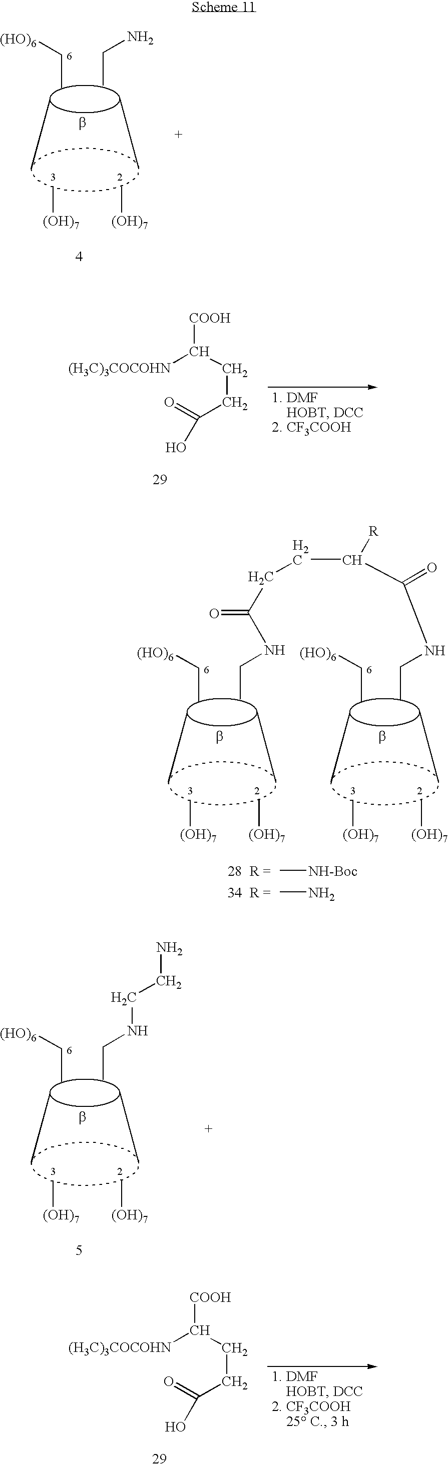

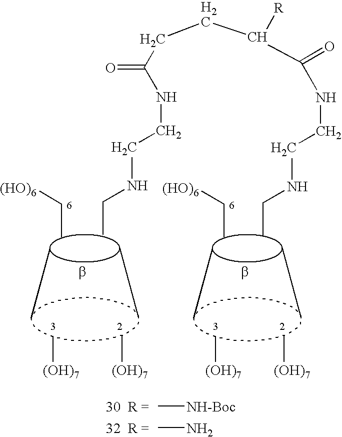

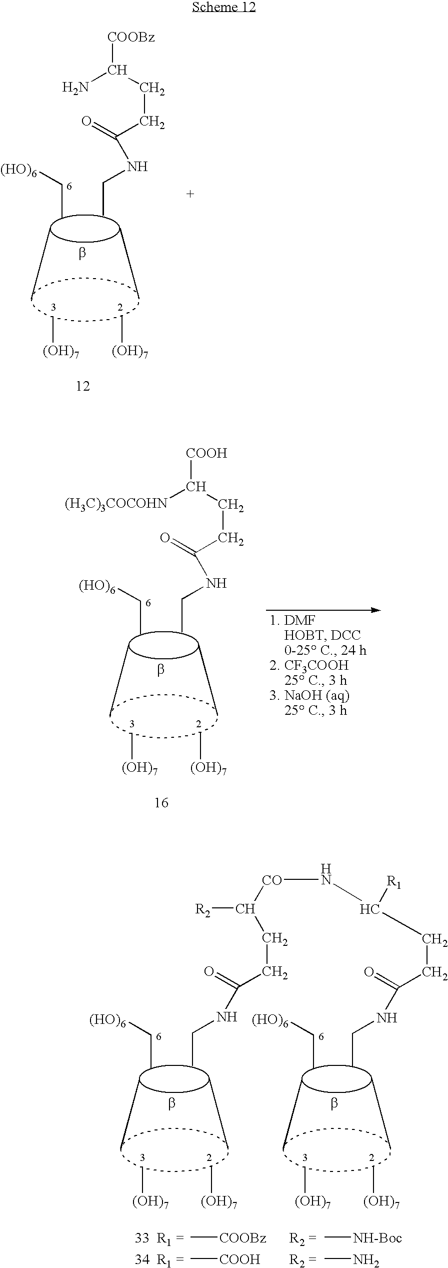

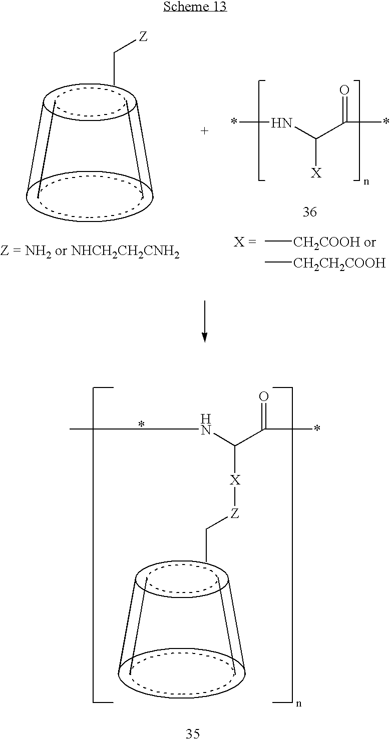

### REFERENCES

Barse B., Kaul P., Banerjee A., Kaul, C. L. and Banerjee, U. C., 2003. “Cyclodextrins: Emerging applications” Chimica Oggi, 21: 48-54.
Dai C., Wang B. and Zhao H., 2005. “Microencapsulation peptide and protein drugs delivery system” Colloids and Surfaces B Biointerfaces, 41 (2-3): 117-120.
Duchene D. 1987. Cyclodextrin and their Industrial Uses. Editions de la Sante, Paris.
International Food Ingredients 2003. “New potential of microencapsulation”, 5: 100-102.
Li J. and Liu D. 2003, “Progress of the Application of beta-Cyclodextrin and Its Derivatives in Analytical Chemistry” Physical Testing and Chemical Analysis Part B Chemical Analysis, 39(6):372-376.
Orriols A., GarciaMontoya E., Minarro M., Perez-Lozano P. SuneNegre J. M. and Tico J. R. 2005. “Microencapsulation: applications, methods and characterization”, Ciencia Y Tecnologia Pharmaceutica, 15(4): 169-176.
Parrot-Lopez H., Djedaini F., Perly B., Coleman A. W., Galons, H. and Miocque M. 1990a. Tetrahedron Lett., 31: 1999-2002.
Parrot-Lopez H., Galons H., Coleman A. W., Djedaini F., Keller N. and Perly B. 1990b. Tetrahedron Asymmetry, 1: 367-370.
Parrot-Lopez H., Galons H., Dupas S., Miocque M. and Tsoucaris G. 1990c Bull. Soc. Chim. Fr., 127: 568-571.
Takahashi K., Ohtasuka Y., Nakada S. and Hattori, K. 1991 J. Incl. Phenom., 10: 63-68.

1-42. (canceled)

43. A cyclodextrin (CD)-containing polymer comprising one or more cyclodextrin residues, wherein said polymer is selected from the group consisting of a peptide, a polypeptide, an oligonucleotide and a polynucleotide or a mixture thereof, said peptide or polypeptide has at least one amino acid residue containing a functional side group and at least one of the cyclodextrin residues is covalently linked to said functional side group of the amino acid residue of said peptide or polypeptide or to the sugar moiety of a nucleotide residue of said oligonucleotide or polynucleotide.

44. A cyclodextrin-containing polymer according to claim 43, comprising one or more cyclodextrin residues, wherein at least one of the cyclodextrin residues is covalently linked to a functional side group of an amino acid residue of a peptide or polypeptide, and said peptide or polypeptide is an all-L, all-D or an L,D-peptide or polypeptide, in which the amino acids may be natural amino acids, non-natural amino acids or chemically modified amino acids.

45. The cyclodextrin-containing polymer according to claim 44, wherein said natural amino acid is selected from the 20 natural amino acids but at least one of the amino acids has a functional side group and is selected from the group consisting of lysine, aspartic acid, glutamic acid, cysteine, serine, threonine, tyrosine and histidine; said said non-natural amino acid is selected from the group consisting of an Nα-methyl amino acid, a Cα-methyl amino acid, a β-methyl amino acid, β-alanine (β-Ala), norvaline (Nva), norleucine (Nle), 4-aminobutyric acid (γ-Abu), 2-aminoisobutyric acid (Aib), ornithine (Orn), 6-aminohexanoic acid (ε-Ahx), hydroxyproline (Hyp), sarcosine, citruline, cysteic acid, statine, aminoadipic acid, homoserine, homocysteine, 2-aminoadipic acid, diaminopropionic (Dap) acid, hydroxylysine, homovaline, homoleucine, TIC, naphthylalanine (Nal), and a ring-methylated or halogenated derivative of Phe; and said chemically modified amino acid is selected from the group consisting of: (a) N-acyl derivatives of the amino terminal or of another free amino group, wherein the acyl group may be an alkanoyl group such as acetyl, hexanoyl, octanoyl or an aroyl group, e.g., benzoyl; (b) esters of the carboxyl terminal or of other free carboxyl or hydroxy group(s); and (c) amides of the carboxyl terminal or of another free carboxyl group(s).

46. The cyclodextrin-containing polymer according to claim 44, wherein said peptide is an oligopeptide of 2-20, preferably, 2-10 amino acid residues, and said polypeptide has 21-10,000, preferably, 100-500 amino acid residues.

47. The cyclodextrin-containing polymer according to claim 46, wherein said oligopeptide is composed of identical amino acid residues, preferably the CD-containing oligopeptides herein identified as 24, 25, 26, and 27.

48. The cyclodextrin-containing polymer according to claim 47, wherein the oligopeptide has two amino acid residues, such as glu-glu or asp-asp or Lys-Lys or Cys-Cys, preferably the CD-containing dipeptides herein identified as 33 and 34.

49. The cyclodextrin-containing polymer according to claim 46, wherein the polypeptide is a homopolypeptide of an amino acid having a functional side group such as polylysine, polyglutamic acid, polyaspartic acid, polycysteine, polyserine, polythreonine or polytyrosine or a random copolymer of different amino acids, wherein at least one of the amino acids has a functional side group.

50. The cyclodextrin-containing polymer according to claim 44, wherein said peptide or polypeptide is further covalently linked to a carbohydrate residue to form a glycopeptide, a glycopolypeptide or a glycoprotein.

51. The cyclodextrin-containing polymer according to claim 44, wherein said polypeptide is a native, preferably inert protein such as albumin, collagen, lectins, hormones or enzymes such as collagenases, matrix metalloproteinases (MMPs) or protein kinases such as Src, v-Src, growth factors, or a protein fragment such as epidermal growth factor (EGF) fragment.

52. The cyclodextrin-containing polymer according to claim 43, wherein the cyclodextrin is selected from α-, β-, γ-cyclodextrin or a combination thereof preferably a β-cyclodextrin or a β-cyclodextrin derivative.

53. The cyclodextrin-containing polymer according to claim 52, containing at least two cyclodextrin residues, wherein the cyclodextrin residues may be identical or different.

54. The cyclodextrin-containing polymer according to claim 43, containing an active ingredient encapsulated within the cavity of said cyclodextrin residues, wherein said active ingredient is a hydrophobic or is a water-insoluble or water-unstable molecule, and is at least one agent having biological activity, an odor agent or a color agent.

55. The cyclodextrin-containing polymer according to claim 54, wherein the active ingredient is further entrapped or embedded (microencapsulated) within the polymer matrix.

56. A composition comprising a cyclodextrin-containing polymer according to claim 43.

57. A pharmaceutical composition comprising a biologically active agent or drug encapsulated into the cyclodextrin cavity and/or entrapped within a cyclodextrin-containing polymer according to claim 43.

58. A cosmetic or dermatological composition comprising an active agent encapsulated and/or entrapped within a cyclodextrin-containing polymer according to claim 43, wherein said encapsulated active agent is a cosmetic or cosmeticeutical agent.

59. A process for producing a cyclodextrin-containing peptide or polypeptide according to claim 44, comprising the steps of:
(i) preparing a modified cyclodextrin by replacement of at least one of the hydroxyl groups with a functional group selected from the group consisting of —NH2, —NH(CH2)mNH2, —SH, —O(CH2)mCOOH, —OC(O)(CH2)mCOOH, —NH(CH2)mCOOH, —OC(O)(CH2)mNH2, —Br, —Cl, —I and —OSO2(c6-C10)aryl, preferably phenyl, wherein m is 1, 2, 3, 4, or 5;
(ii) covalently linking the modified cyclodextrin to a functional side group of a diprotected amino acid residue to form a cyclodextrin-amino acid derivative; and
(iii) polymerizing said cyclodextrin-amino acid derivative to obtain the desired cyclodextrin-containing polypeptide.

60. A process for producing a cyclodextrin-containing peptide or polypeptide according to claim 44, comprising the steps of:
(i) preparing a modified cyclodextrin by replacement of at least one of the hydroxyl groups with a functional group selected from the group consisting of —NH2, —NH(CH2)mNH2, —SH, —O(CH2)mCOOH, —OC(O)(CH2)mCOOH, —NH(CH2)mCOOH, —OC(O)(CH2)mNH₂, —Br, —Cl, —I and —OSO₂(c6-C10)aryl, preferably phenyl, wherein m is 1, 2, 3, 4, or 5; and
(ii) covalently grafting the modified cyclodextrin directly to the side chain of the peptide or polypeptide.

61. A process for producing a cyclodextrin-containing dipeptide according to claim 44, comprising the steps of:
(i) preparing a modified cyclodextrin by replacement of at least one of the hydroxyl groups with a functional group selected from the group consisting of —NH2, —NH(CH2)mNH2, —SH, O(CH2)mCOOH, OC(O)(CH2)mCOOH, —NH(CH2)mCOOH, —OC(O)(CH2)mNH2, —Br, —Cl, - and, —OSO2(c6-C10)aryl, preferably phenyl, wherein m is 1, 2, 3, 4, or 5;
(ii) covalently linking the modified cyclodextrin to a side functional group of a diprotected amino acid residue to form a CD-amino acid derivative (a); and
(iii) covalently linking another modified CD to a side functional group of a diprotected amino acid residue to form a second CD-amino acid derivative (b); and
(iv) coupling the free α-amino of the CD-amino acid compound (a) with the free α-carboxy of the CD-amino acid compound (b) to obtain the desired CD-containing dipeptide.

62. A carrier consisting of a cyclodextrin-containing polymer according to claim 43, for controlled release of water-insoluble or unstable active agents.

63. A composition for controlled release of water-insoluble or unstable active ingredient(s) comprising nanoparticles of said active ingredient(s) encapsulated and/or entrapped within a CD-containing polymer according to claim 43.

64. A method for combined microencapsulation and molecular encapsulation of an active agent in a sole carrier which comprises reacting said active agent with a CD-containing polymer according to claim 43, whereby the active agent is both encapsulated and entrapped within said CD-containing polymer.

65. A di-CD-amino acid derivative comprising two residues of a native or modified cyclodextrin covalently linked to one molecule of an amino acid selected from glutamic acid, aspartic acid or lysine, preferably the derivatives herein identified as 28, 30, 31 and 32.

66. A di-CD-amino acid derivative according to claim 65, containing an active agent encapsulated within the cavities of the cyclodextrin residues and within the cavity or pouch formed by the amino acid and the two CD residues.

67. A composition comprising a di-CD-amino acid derivative according to claim 65.

68. A pharmaceutical composition comprising a di-CD-amino acid derivative according to claim 65, wherein said encapsulated active agent is a biologically active agent or drug.

69. A cosmetic or dermatological composition comprising a di-CD-amino acid derivative according to claim 65, wherein said encapsulated active agent is a cosmetic or cosmeticeutical agent.

70. A mono(6-aminoethylamino-6-deoxy)cyclodextrin covalently linked via the 6-position CD-NH—CH2-CH2-NH— group to the functional side group of an α-amino acid selected from the group consisting of aspartic acid, glutamic acid, lysine, tyrosine, cysteine, serine, threonine and histidine, preferably the compounds herein identified as 10, 11, 14, 15, 18 and 19.

71. A mono(6-amino-6-deoxy)cyclodextrin covalently linked via the 6-position CD-NH— group to the functional side group of an α-amino acid selected from the group consisting of aspartic acid, glutamic acid, lysine, tyrosine, cysteine, serine, threonine and histidine, wherein the α-amino or both the α-amino and the α-carboxy groups are protected, preferably the compounds herein identified as 6, 8, 16, and 17.
